(19) United States
(12) Patent Application Publication
(10) Pub. No.: US 2015/0313481 A1
(43) Pub. Date: Nov. 5, 2015

(54) CARDIOVASCULAR SCREENING DIAGNOSTIC AND MONITORING SYSTEM AND METHOD

(71) Applicant: M.I. MEDICAL INCENTIVE LTD., Haifa (IL)

(72) Inventors: Yehonatan TIROSH, Tel Aviv (IL); Daniel B LEVIN, Haifa (IL)

(73) Assignee: M.I. MEDICAL INCENTIVE LTD, Haifa (IL)

(21) Appl. No.: 14/655,280

(22) PCT Filed: Dec. 31, 2013
(86) PCT No.: PCT/IL13/51100

§ 371 Date: Jun. 24, 2015

Related U.S. Application Data
(60) Provisional application No. 61/747,716, filed on Dec. 31, 2012.

(51) Int. Cl. A61B 5/02; A61B 5/11; A61B 5/00; A61B 5/0205

(52) U.S. Cl. CPC A61B 5/02028; A61B 5/02055; A61B 5/1118; A61B 5/4884; A61B 5/7246; A61B 5/7282; A61B 5/681; A61B 5/6895

(57) ABSTRACT

A system for providing an indication of at least LVD (Left Ventricular Dysfunction) including at least one temperature sensor providing an output indication based on skin temperature at at least one location on a person at a plurality of given times, at least one body activity sensor providing an output indication of at least termination of body activity, a time/temperature ascertainer operative to receive inputs from the at least one temperature sensor and from the at least one body activity sensor to provide output indications of the skin temperature at termination of body activity and thereafter and a correlator operative to correlate the output indications of the skin temperature at termination of body activity and thereafter with established clinical data relating changes in skin temperature at termination of body activity and thereafter to existence of at least LVD, the correlator providing at least an output indication of at least LVD.

### REFERENCE TO RELATED APPLICATIONS

Reference is hereby made to U.S. Provisional Patent Application Ser. No. 61/747,716, filed Dec. 31, 2012 and entitled SYSTEM AND METHOD FOR DETERMINING CARDIOVASCULAR CONDITION, the disclosure of which is hereby incorporated by reference and priority of which is hereby claimed pursuant to 37 CFR 1.78(a) (4) and (5)(i).

### FIELD OF THE INVENTION

The present invention relates to medical diagnostic systems and methods generally and more particularly to diagnosis of LVD (Left Ventricular Dysfunction).

### BACKGROUND OF THE INVENTION

Various types of systems and methods for cardiac function diagnosis are known in the art.

### SUMMARY OF THE INVENTION

The present invention seeks to provide improved medical diagnostic systems for the diagnosis of LVD (Left Ventricular Dysfunction).

There is thus provided in accordance with a preferred embodiment of the present invention a system for providing an indication of at least LVD (Left Ventricular Dysfunction), the system including at least one temperature sensor providing an output indication based on skin temperature at at least one location on a person at a plurality of given times, at least one body activity sensor providing an output indication of at least termination of body activity, a time/temperature ascertainer operative to receive inputs from the at least one temperature sensor and from the at least one body activity sensor to provide output indications of the skin temperature at termination of body activity and thereafter and a correlator operative to correlate the output indications of the skin temperature at termination of body activity and thereafter with established clinical data relating changes in skin temperature at termination of body activity and thereafter to existence of at least LVD, the correlator providing at least an output indication of at least LVD.

Preferably, the at least one temperature sensor and the at least one body activity sensor respectively measure temperature and body activity at two distinct regions of a person's body. Alternatively, the at least one temperature sensor and the at least one body activity sensor respectively measure temperature and body activity at a single region of a person's body.

In accordance with a preferred embodiment of the present invention the at least one temperature sensor and the at least one body activity sensor respectively measure temperature and body activity such that the temperature represents skin temperature at a body region which is less active than a region which is principally undergoing body activity.

Preferably, the at least one body activity sensor is embodied in a treadmill. Additionally of alternatively, the temperature sensor measures skin temperature on a person's wrist.

In accordance with a preferred embodiment of the present invention body activity sensor is mounted on a portion of the person's body which is undergoing physical exertion while the temperature sensor is mounted on a portion of the person's body other than that portion undergoing physical exertion.

Preferably, physical exertion of the person is measured from a starting point in time designated time A at which the person is standing and at rest, the onset of physical exertion begins at a point in time designated B and the physical exertion is terminated at a point in time designated C. Additionally, a time separation between points A and B is approximately 2 minutes, a time separation between time points B and C is approximately 4 minutes and a further measuring point in time, designated time point D, is established at approximately 2.3 minutes following time point C.

In accordance with a preferred embodiment of the present invention measured differential skin temperature relative to point C (MDST(−C)) increases from time point C to time point D for a non-LVD person. Additionally or alternatively, measured differential skin temperature relative to point C (MDST(−C)) decreases from time point C to time point D for an LVD person.

Preferably, the system also includes an ejection fraction calculator operative to ascertain the ejection fraction (EF) for the person.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person, measured differential skin temperature relative to point C (MDST(−C)) decreases from time point C to time point D for an LVD person, and the ejection fraction calculator employs an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD

Where K₁-K₉ are constants, MDST(D−C) is the Measured Differential Skin Temperature relative to point C at point D, A is Age in Years, MF is 0 for males and 1 for females, W is Weight in Kilograms, HT is Height in Centimeters, DTDE is Distance in meters Traveled during Physical Exertion, DPEM is Duration of Physical Exertion in Minutes and LVD is 0 for non-LVD and 1 for LVD.

Preferably, K₁ is approximately 26, K₂ is approximately −1.5, K₃ is approximately −0.1, K₄ is approximately 1.93, K₅ is approximately −0.3, K₆ is approximately 0.3, K₇ is approximately −0.03, K₈ is approximately 2.6 and K₉ is approximately −30.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person,

measured differential skin temperature relative to point C (MDST(−C)) decreases from time point C to time point D for an LVD person and the ejection fraction calculator employs an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD+K₁₀×SBP+K₁₁×DBP+K₁₂×TEMP

Where K₁-K₁₂ are constants, MDST(D−C) is the Measured differential skin temperature relative to point C at point D, A is Age in Years, MF is 0 for males and 1 for females, W is Weight in Kilograms, HT is Height in Centimeters, DTDE is Distance in meters traveled during Physical Exertion, DPEM is Duration of Physical Exertion in Minutes, LVD is 0 for non-LVD and 1 for LVD, SBP is Systolic Blood Pressure in mm HG, DBP is Diastolic Blood Pressure in mm HG and TEMP is Oral Temperature in ° C.

Preferably, K₁ is approximately −26, K₂ is approximately −7, K₃ is approximately −0.05, K₄ is approximately 1.3, K₅ is approximately −0.2, K₆ is approximately 0.2, K₇ is approximately −0.05, K₈ is approximately 3.6, K₉ is approximately −32, K₁₀ is approximately 0.05, K₁₁ is approximately 0.1 and K₁₂ is approximately 1.3.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person, measured differential skin temperature relative to point C (MDST(−C)) decreases from time point C to time point D for an LVD person and the ejection fraction calculator employs an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD+K₁₀×SBP+K₁₁×DBP+K₁₂×TEMP+K₁₃×HRC/HRD

Where K₁-K₁₃ are constants, MDST(D−C) is the Measured Differential skin temperature relative to point C at point D, A is Age in Years, MF is 0 for males and 1 for females, W is Weight in Kilograms, HT is Height in Centimeters, DTDE is Distance in meters Traveled during physical Exertion, DPEM is Duration of Physical Exertion in Minutes, LVD is 0 for non-LVD and 1 for LVD, SBP is Systolic Blood Pressure in mm HG, DBP is Diastolic Blood Pressure in mm HG, TEMP is Oral Temperature in ° C., HRC is Heart Rate at time point C in Beats Per Minute (BPM) and HRD is Heart Rate at time point D in BPM.

Preferably, K₁ is approximately 10, K₂ is approximately −3, K₃ is approximately −0.1, K₄ is approximately −0.2, K₅ is approximately −0.2, K₆ is approximately 0.2, K₇ is approximately −0.05, K₈ is approximately 3.3, K₉ is approximately −31, K₁₀ is approximately 0.1, K₁₁ is approximately 0.01, K₁₂ is approximately 0.4 and K₁₃ is approximately −1.

In accordance with a preferred embodiment of the present invention the body activity sensor provides outputs indicating ONSET OF PHYSICAL EXERTION (DOPE) (Time Point B), TERMINATION OF PHYSICAL EXERTION (TOPE) (Time Point C) and DISTANCE TRAVELED DURING PHYSICAL EXERTION (DTDE). Additionally, the system also includes a minimum exertion level calculator receiving the outputs of the body activity sensor and providing an output indicating whether a minimum threshold for physical exertion has been exceeded between the OOPE and the TOPE.

In accordance with a preferred embodiment of the present invention body activity of the person is measured from a starting point in time designated time E at which the person is sitting and at rest and the body activity terminates at a point in time designated time F. Additionally, a time separation between points E and F is approximately 2 minutes, a time separation between time point F and a reference time point G is approximately 3 minutes and at least one of three further measuring points in time, designated time points H1, H2 & H3, is established at approximately 2 minutes, 3 minutes and 6 minutes following time point G.

Preferably, at least two of three further measuring points in time, designated time points H1, H2 & H3, are established at approximately 2 minutes, 3 minutes and 6 minutes following time point G. Additionally, three further measuring points in time, designated time points H1, H2 & H3, are established at approximately 2 minutes, 3 minutes and 6 minutes following time point G.

In accordance with a preferred embodiment of the present invention a measured differential skin temperature (MDST(−G)) decreases more significantly following time point G for an LVD person than for a non-LVD person.

Preferably, the system also includes an ejection fraction calculator operative to ascertain the ejection fraction (EF) for the person.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) is calculated and the ejection fraction calculator employs an algorithm of the following general form:

Ejection Fraction(EF)(%)=K₁+K₂×MDST(H2−G)+K₃×A+K₄×MF+K₅×W±K₆×HT+K₇×SBP+K₈×DBP+K₉×TEMP

Where K₁-K₉ are constants, MDST(H2−G) is the Measured Differential skin temperature relative to point G at point H2, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG and TEMP is Oral Temperature in ° C.

Preferably, K₁ is approximately −1694, K₂ is approximately 100, K₃ is approximately 0.59, K₄ is approximately 44.2, K₅ is approximately −1.71, K₆ is approximately 2.22, K₇ is approximately −1.41, K₈ is approximately −0.05 and K₉ is approximately 44.3.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) is calculated and the ejection fraction calculator employs an algorithm of the following general form:

Ejection Fraction(EF)(%)=K₁+K₂×MDST(H3−G)+K₃×A+K₄×MF+K₅×W±K₆×HT+K₇×SBP+K₈×DBP+K₉×TEMP

Where K₁-K₉ are constants, MDST(H3−G) is the Measured Differential skin temperature relative to point G at point H3, A is Age in Years, MF is 0 for males and 1 for females, W is Weight in Kilograms, HT is Height in Centimeters, LVD is 0 for non-LVD and 1 for LVD, SBP is Systolic Blood Pressure in mm HG, DBP is Diastolic Blood Pressure in mm HG and TEMP is Oral Temperature in ° C.

Preferably, K₁ is approximately −1065, K₂ is approximately 55.6, K₃ is approximately 0.36, K₄ is approximately 34.1, K₅ is approximately 1.37, K₆ is approximately 1.58, K₇ is approximately −1.10, K₈ is approximately −0.07 and K₉ is approximately 29.0.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) decreases more significantly following time point G for an LVD person than for a non-LVD person and the ejection fraction calculator employs an algorithm of the following general form:

Ejection Fraction(EF)(%)=K₁+K₂×MDST(H1−G)+K₃×A+K₄×MF+K₅×W±K₆×HT+K₇×SBP+K₈×DBP+K₉×TEMP+K₁₀×LVD

Where K₁-K₉ are constants, MDST(H1−G) is the Measured Differential skin temperature relative to point G at point H1, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG, TEMP is Oral Temperature in ° C. and LVD is 0 for non-LVD and 1 for LVD.

Preferably, K₁ is approximately −192, K₂ is approximately 35.5, K₃ is approximately 0.11, K₄ is approximately 4.05, K₅ is approximately 0.33, K₆ is approximately 0.30, K₇ is approximately −0.11, K₈ is approximately 0.03, K₉ is approximately 6.32 and K₁₀ is approximately −26.0.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G and a measured differential skin temperature relative to point G (MDST(−G)) decreases more significantly following time point G for an LVD person than for a non-LVD person and the ejection fraction calculator employs an algorithm of the following general form:

Ejection Fraction(EF)(%)=K₁+K₂×MDST(H3−G)+K₃×A+K₄×MF+K₅×W±K₆×HT+K₇×SBP+K₈×DBP+K₉×TEMP+K₁₀×LVD

Where K₁-K₉ are constants, MDST(H3−G) is the Measured Differential skin temperature relative to point G at point H3, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG, TEMP is Oral Temperature in ° C. and LVD is 0 for non-LVD and 1 for LVD.

Preferably, K₁ is approximately −85.3, K₂ is approximately 14.4, K₃ is approximately 0.07, K₄ is approximately 3.04, K₅ is approximately −0.24, K₆ is approximately 0.19, K₇ is approximately −0.10, K₈ is approximately 0.05, K₉ is approximately 3.77 and K₁₀ is approximately −24.7.

In accordance with a preferred embodiment of the present invention the body activity sensor provides outputs indicating ONSET OF POSITION CHANGE (OOPC), TERMINATION OF POSITION CHANGE (TOPC) (Time Point F) and CHANGE IN POSITION (CIP). Additionally, the system also includes a body position change calculator receiving the outputs of the body activity sensor and providing an output indicating whether a qualifying position change has been performed between the OOPC and the TOPC as well as the TYPE OF POSITION CHANGE (TYPC).

There is also provided in accordance with another preferred embodiment of the present invention a method for providing an indication of at least LVD (Left Ventricular Dysfunction), the method including sensing a skin temperature of a subject at at least one location on a person at a plurality of given times, providing a plurality of skin temperature output indications based on the sensing, sensing body activity of the subject and providing an output indication of at least termination of the body activity, ascertaining skin temperature of the subject at the termination of body activity and thereafter based on the plurality of skin temperature output indications and the output indication of at least termination of the body activity, correlating the skin temperature of the subject at the termination of body activity and thereafter with established clinical data relating changes in skin temperature at the termination of body activity and thereafter to existence of at least LVD and providing at least an output indication of at least LVD.

Preferably, the sensing a skin temperature and the sensing body activity respectively include sensing skin temperature and sensing body activity at two distinct regions of a person's body. Alternatively, the sensing a skin temperature and the sensing body activity respectively include sensing skin temperature and sensing body activity at a single region of a person's body.

In accordance with a preferred embodiment of the present invention the sensing a skin temperature and the sensing body activity respectively include sensing skin temperature at a body region which is less active than a region which is principally undergoing body activity.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes and a further measuring point in time, designated time point D, is established at approximately 2.3 minutes following time point C.

Preferably, the output indication of at least LVD indicates the absence of LVD when measured differential skin temperature relative to point C (MDST(−C)) increases from time point C to time point D. Additionally or alternatively, the output indication of at least LVD indicates the presence of LVD when measured differential skin temperature relative to point C (MDST(−C)) decreases from time point C to time point D.

In accordance with a preferred embodiment of the present invention the method also includes ascertaining an ejection fraction (EF) for the subject.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person, measured differential skin temperature relative to point C (MDST(−C)) decreases from time point C to time point D for an LVD person and the ascertaining an ejection fraction includes employing an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD

Where K₁-K₉ are constants, MDST(D−C) is the Measured Differential skin temperature relative to point C at point D, A is Age in Years, MF is 0 for males and 1 for females, W is Weight in Kilograms, HT is Height in Centimeters, DTDE is Distance in meters Traveled during Physical Exertion, DPEM is Duration of Physical Exertion in Minutes and LVD is 0 for non-LVD and 1 for LVD.

Preferably, K₁ is approximately 26, K₂ is approximately −1.5, K₃ is approximately −0.1, K₄ is approximately 1.93, K₅ is approximately −0.3, K₆ is approximately 0.3, K₇ is approximately −0.03, K₈ is approximately 2.6 and K₉ is approximately −30.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person, measured differential skin temperature relative to point C (MDST(−C)) decreases from time point C to time point D for an LVD person and the ascertaining an ejection fraction includes employing an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD+K₁₀×SBP+K₁₁×DBP+K₁₂×TEMP

Where K₁-K₁₂ are constants, MDST(D−C) is the Measured differential skin temperature relative to point C at point D, A is Age in Years, MF is 0 for males and 1 for females, W is Weight in Kilograms, HT is Height in Centimeters, DTDE is Distance in meters traveled during Physical Exertion, DPEM is Duration of Physical Exertion in Minutes, LVD is 0 for non-LVD and 1 for LVD, SBP is Systolic Blood Pressure in mm HG, DBP is Diastolic Blood Pressure in mm HG and TEMP is Oral Temperature in ° C.

Preferably, K₁ is approximately −26, K₂ is approximately −7, K₃ is approximately −0.05, K₄ is approximately 1.3, K₅ is approximately −0.2, K₆ is approximately 0.2, K₇ is approximately −0.05, K₈ is approximately 3.6, K₉ is approximately −32, K₁₀ is approximately 0.05, K₁₁ is approximately 0.1 and K₁₂ is approximately 1.3.

In accordance with a preferred embodiment of the present invention physical exertion of the person is measured from a starting point in time designated time A at which the person is standing and at rest, the onset of physical exertion begins at a point in time designated B, the physical exertion is terminated at a point in time designated C, a time separation between points A and B is approximately 2 minutes, a time separation between time points B and C is approximately 4 minutes, a further measuring point in time, designated time point D, is established at approximately 2.3 minutes following time point C, measured differential skin temperature relative to point C (MDST(−C)) increases from time point C to time point D for a non-LVD person, measured differential skin temperature relative to point C (MDST(−C)) decreases from time point C to time point D for an LVD person, and the ascertaining an ejection fraction includes employing an algorithm of the following general form:

Ejection Fraction(EF)(%)=K₁+K₂×MDST(D−C)+K₃×A+K₄×MF+K₅×W±K₆×HT+K₇×DTDE+K₈×DPEM+K₉×LVD+K₁₀×SBP+K₁₁×DBP+K₁₂×TEMP+K₁₃×HRC/HRD

Where K₁-K₁₃ are constants, MDST(D−C) is the Measured Differential skin temperature relative to point C at point D, A is Age in Years, MF is 0 for males and 1 for females, W is Weight in Kilograms, HT is Height in Centimeters, DTDE is Distance in meters Traveled during physical Exertion, DPEM is Duration of Physical Exertion in Minutes, LVD is 0 for non-LVD and 1 for LVD, SBP is Systolic Blood Pressure in mm HG, DBP is Diastolic Blood Pressure in mm HG, TEMP is Oral Temperature in ° C., HRC is Heart Rate at time point C in Beats Per Minute (BPM) and HRD is Heart Rate at time point D in BPM.

Preferably, K₁ is approximately 10, K₂ is approximately −3, K₃ is approximately −0.1, K₄ is approximately −0.2, K₅ is approximately −0.2, K₆ is approximately 0.2, K₇ is approximately −0.05, K₈ is approximately 3.3, K₉ is approximately −31, K₁₀ is approximately 0.1, K₁₁ is approximately 0.01, K₁₂ is approximately 0.4 and K₁₃ is approximately −1.

In accordance with a preferred embodiment of the present invention the body activity of the person is measured from a starting point in time designated time E at which the person is sitting and at rest, the body activity terminates at a point in time designated time F, a time separation between points E and F is approximately 2 minutes, a time separation between time point F and a later reference time point G is approximately 3 minutes, and at least one of three further measuring points in time, designated time points H1, H2 & H3, is established at approximately 2 minutes, 3 minutes and 6 minutes following time point G.

Preferably, a measured differential skin temperature relative to point G (MDST(−G)) decreases more significantly following time point G for an LVD person than for a non-LVD person.

In accordance with a preferred embodiment of the present invention the output indication of at least LVD indicates absence of LVD when measured differential skin temperature relative to point G (MDST(−G)) between time point G and at least one of time points H1, H2 and H3 is higher than a respective predetermined threshold. Additionally or alternatively, the output indication of at least LVD indicates absence of LVD when measured differential skin temperature relative to point G (MDST(−G)) decreases at a lower rate than a respective predetermined threshold from time point G to at least one of time points H1, H2 and H3.

Preferably, the output indication of at least LVD indicates presence of LVD when measured differential skin temperature relative to point G (MDST(−G)) decreases at a higher rate than a respective predetermined threshold from time point G to at least one of time points H1, H2 and H3. Additionally or alternatively, the output indication of at least LVD indicates presence of LVD when measured differential skin temperature relative to point G (MDST(−G)) between time point G and at least one of time points H1, H2 and H3 is lower than a respective predetermined threshold.

In accordance with a preferred embodiment of the present invention the method also includes ascertaining an ejection fraction (EF) for the subject.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) is calculated and the ascertaining an ejection fraction includes employing an algorithm of the following general form:

Ejection Fraction(EF)(%)=K₁+K₂×MDST(H2−G)+K₃×A+K₄×MF+K₅×W±K₆×HT+K₇×SBP+K₈×DBP+K₉×TEMP

Where K₁-K₉ are constants, MDST(H2−G) is the Measured Differential skin temperature relative to point G at point H2, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG and TEMP is Oral Temperature in ° C.

Preferably, K₁ is approximately −1694, K₂ is approximately 100, K₃ is approximately 0.59, K₄ is approximately 44.2, K₅ is approximately 1.71, K₆ is approximately 2.22, K₇ is approximately −1.41, K₈ is approximately −0.05 and K₉ is approximately 44.3.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) is calculated and the ascertaining an ejection fraction includes employing an algorithm of the following general form:

Ejection Fraction(EF)(%)=K₁+K₂×MDST(H3−G)+K₃×A+K₄×MF+K₅×W±K₆×HT+K₇×SBP+K₈×DBP+K₉×TEMP

Where K₁-K₉ are constants, MDST(H3−G) is the Measured Differential skin temperature relative to point G at point H3, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG and TEMP is Oral Temperature in ° C.

Preferably, K₁ is approximately −1065, K₂ is approximately 55.6, K₃ is approximately 0.36, K₄ is approximately 34.1, K₅ is approximately 1.37, K₆ is approximately 1.58, K₇ is approximately −1.10, K₈ is approximately −0.07 and K₉ is approximately 29.0.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) decreases more significantly following time point G for an LVD person than for a non-LVD person and the ascertaining an ejection fraction includes employing an algorithm of the following general form:

Ejection Fraction(EF)(%)=K₁+K₂×MDST(H1−G)+K₃×A+K₄×MF+K₅×W±K₆×HT+K₇×SBP+K₈×DBP+K₉×TEMP+K₁₀×LVD

Where K₁-K₁₀ are constants, MDST(H1−G) is the Measured Differential skin temperature relative to point G at point H1, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG, TEMP is Oral Temperature in ° C. and LVD is 0 for non-LVD and 1 for LVD.

Preferably, K₁ is approximately −192, K₂ is approximately 35.5, K₃ is approximately 0.11, K₄ is approximately 4.05, K₅ is approximately 0.33, K₆ is approximately 0.30, K₇ is approximately −0.11, K₈ is approximately 0.03, K₉ is approximately 6.32 and K₁₀ is approximately −26.0.

In accordance with a preferred embodiment of the present invention body activity of the person is measured from a starting point in time designated time E at which the person is sitting and at rest, the body activity terminates at a point in time designated time F, a time separation between points E and F is approximately 2 minutes, a time separation between time point F and a reference time point G is approximately 3 minutes, at least one of three further measuring points in time, designated time points H1, H2 & H3, is established at approximately 2 minutes, 3 minutes and 6 minutes following time point G, a measured differential skin temperature relative to point G (MDST(−G)) decreases more significantly following time point G for an LVD person than for a non-LVD person and the ascertaining an ejection fraction includes employing an algorithm of the following general form:

Ejection Fraction(EF)(%)=K₁+K₂×MDST(H3−G)+K₃×A+K₄×MF+K₅×W±K₆×HT+K₇×SBP+K₈×DBP+K₉×TEMP+K₁₀×LVD

Where K₁-K₁₀ are constants, MDST(H3−G) is the Measured Differential skin temperature relative to point G at point H3, A is Age in Years, MF is 0 for males and 1 for females, W is Weight in Kilograms, HT is Height in Centimeters, SBP is Systolic Blood Pressure in mm HG, DBP is Diastolic Blood Pressure in mm HG, TEMP is Oral Temperature in ° C. and LVD is 0 for non-LVD and 1 for LVD.

Preferably, K₁ is approximately −85.3, K₂ is approximately 14.4, K₃ is approximately 0.07, K₄ is approximately 3.04, K₅ is approximately 0.24, K₆ is approximately 0.19, K₇ is approximately −0.10, K₈ is approximately 0.05, K₉ is approximately 3.77 and K₁₀ is approximately −24.7.

### BRIEF DESCRIPTION OF THE DRAWINGS

The present invention will be understood and appreciated more fully from the following detailed description, taken in conjunction with the drawings in which:

FIG. 1 is a simplified illustration of a system which produces an output indication of change in skin temperature as a time function of physical exertion for a typical person and provides an indication of at least LVD (Left Ventricular Dysfunction) in accordance with a preferred embodiment of the present invention;

FIG. 2 is a simplified illustration of the value of Measured Differential Skin Temperature relative to point C at point D (MDST(D−C)) for a given individual overlaid on a typical graph of MDST(D−C) vs. ejection fraction derived from multiple subjects, which is useful for initial screening of the individual using the system of FIG. 1;

FIG. 3 is a simplified functional block diagram of the system of FIG. 1;

FIG. 4 is a simplified illustration of the values of MDST(D−C) for a given individual monitored on multiple occasions, which is useful for monitoring of the individual using the system of FIG. 1;

FIG. 5 is a simplified flowchart illustrating operation of the system of FIGS. 1-3 for screening;

FIG. 6 is a simplified flowchart illustrating operation of the system of FIGS. 1-4 for EF calculation useful in diagnosis and monitoring;

FIG. 7 is a simplified diagram showing experimental MDST(−C) data for non-LVD subjects and LVD subjects;

FIG. 8 is a simplified diagram showing experimental MDST(−C) data for non-LVD subjects and LVD subjects indicating standard deviations;

FIG. 9 is a simplified illustration of a system which produces an output indication of change in skin temperature as a time function of physical exertion for a typical person and provides an indication of at least LVD (Left Ventricular Dysfunction) in accordance with another preferred embodiment of the present invention;

FIG. 10 is a simplified illustration of the value of Measured Differential Skin Temperature at points H1, H2 and H3 (MDST(H1−G), MDST(H2−G), MDST(H3−G), respectively) for a given individual overlaid on a typical graph of MDST(H1−G), MDST(H2−G), MDST(H3−G) vs. ejection fraction derived from multiple subjects, which is useful for initial screening of the individual using the system of FIG. 9;

FIG. 11 is a simplified functional block diagram of the system of FIG. 9;

FIG. 12 is a simplified illustration of the values of MDST(H1−G) for a given individual monitored on multiple occasions, which is useful for monitoring of the individual using the system of FIG. 9;

FIG. 13 is a simplified flowchart illustrating operation of the system of FIGS. 9-11 for screening;

FIG. 14 is a simplified flowchart illustrating operation of the system of FIGS. 9-12 for EF calculation useful in diagnosis and monitoring;

FIG. 15 is a simplified diagram showing experimental MDST(−G) data for non-LVD subjects and LVD subjects using the system of FIG. 9; and

FIG. 16 is a simplified diagram showing experimental MDST(−G) data for non-LVD subjects and LVD subjects indicating standard deviations using the system of FIG. 9.

### DETAILED DESCRIPTION OF PREFERRED EMBODIMENTS

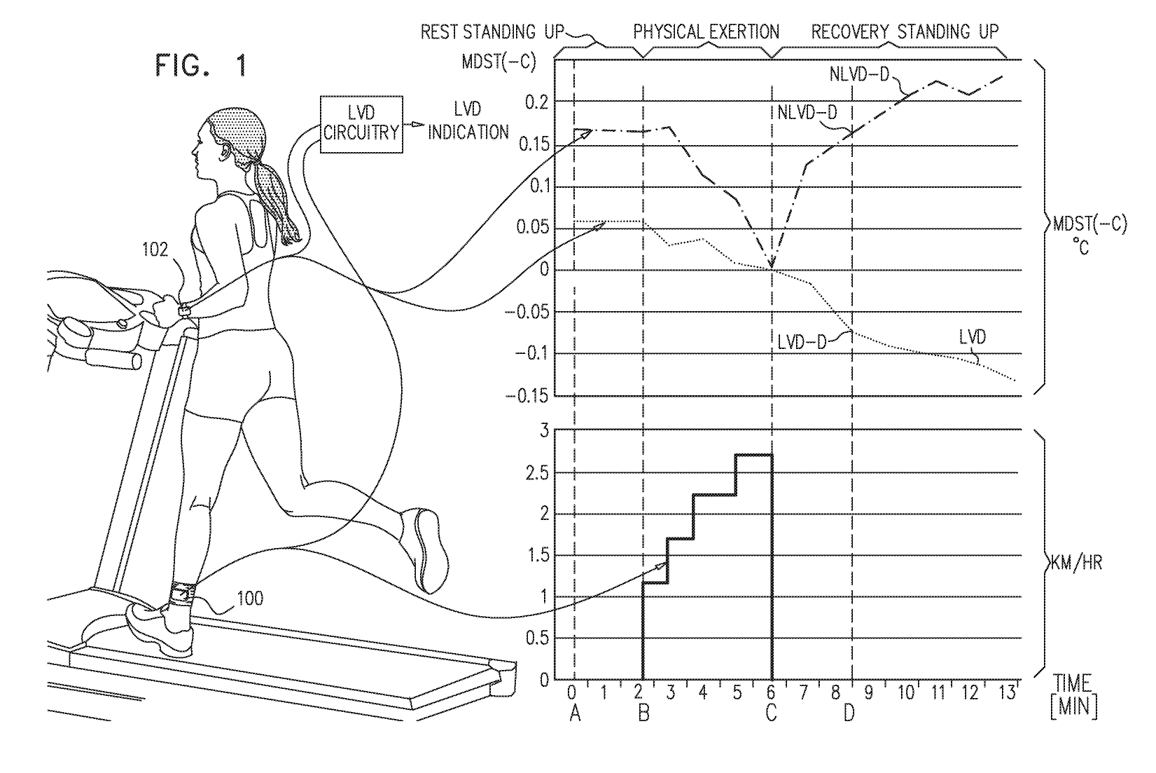

Reference is now made to FIG. 1, which is a simplified illustration of a system which produces an output indication of change in skin temperature as a time function of physical exertion for a typical person and provides an indication of at least LVD (Left Ventricular Dysfunction) in accordance with a preferred embodiment of the present invention.

As seen in FIG. 1, a person, herein sometimes referred to as an individual, is shown undergoing a regimen of timed physical exertion, here, for example, running on a treadmill. The physical exertion of the person is measured by any suitable motion sensor 100, such as a DRM-4000 motion sensor commercially available from Honeywell. The skin temperature of the person is simultaneously measured by a temperature sensor 102, such as an ADT 7420 temperature sensor, commercially available from Analog Devices. The motion sensor 100 is preferably mounted on a portion of the person's body which is undergoing physical exertion, such as the leg of the person, while the temperature sensor 102 is preferably mounted on a portion of the person's body other than that portion undergoing physical exertion, preferably the left wrist of the person.

Considering now the output of the motion sensor 100, it is seen that the physical exertion of the person is measured from a starting point in time, time 0, designated A at which the person is standing and at rest and the onset of physical exertion begins at a point of time designated B and increases in steps, typically to 2.7 km/hr. The physical exertion is terminated at a time point designated C.

The time separation between points A and B is typically and preferably 2 minutes, the time separation between time points B and C is typically and preferably 4 minutes and a further measuring point in time, designated time point D, is established at typically and preferably 2.3 minutes following time point C.

Considering now the output of the temperature sensor 102, it is noted that the graph indicates the difference calculated by subtracting the skin temperature at time point C from the sensed skin temperature at a given time on the graph. The graph of the output of temperature sensor 102 is thus appreciated to be a computed graph which is only provided following time point C.

It is seen that for a non-LVD individual, the measured skin temperature minus the measured skin temperature at time point C, herein designated by reference MDST(−C) (Measured differential skin temperature relative to point C) is typically approximately 0.15° C. between time points A and B and then falls, approximately one minute after time point B generally linearly to zero at time point C. For a typical non-LVD individual, immediately following termination of physical exertion at time point C, the MDST(−C) increases as shown to time point D and typically therebeyond. The MDST(−C) for a non-LVD individual is designated in FIG. 1 by NLVD.

It is seen that for an individual suffering from LVD, the measured skin temperature minus the measured skin temperature at time point C, herein designated by reference MDST(−C) (Measured differential skin temperature relative to point C) is typically approximately 0.05° C. between time points A and B and then falls after time point B to zero at time point C. For a typical individual suffering from LVD, following termination of physical exertion at time point C, the MDST(−C) continues to decrease as shown to time point D and typically therebeyond. The MDST(−C) for an LVD individual is designated in FIG. 1 by LVD.

Appreciation of utilization of the foregoing distinction between MDST(−C) for non-LVD individuals and for LVD individuals are particular features of the present invention.

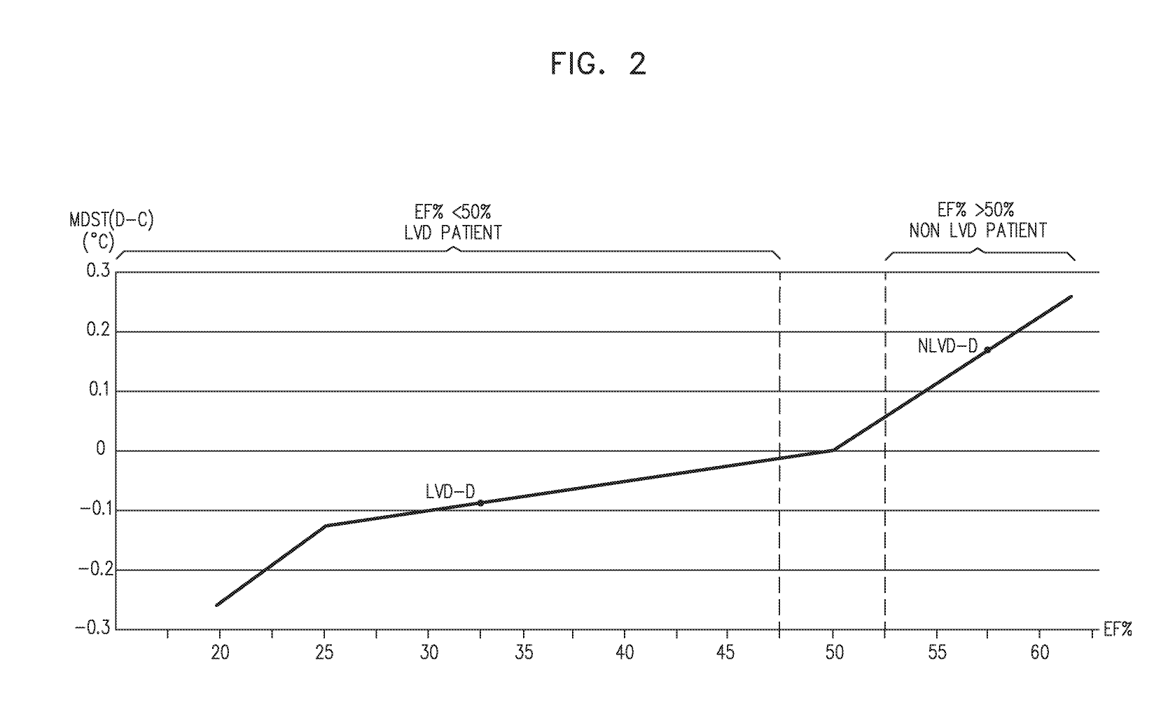

Reference is now made to FIG. 2, which is a simplified illustration of the value of MDST(D−C) for a given individual overlaid on a typical graph of MDST(D−C) vs. ejection fraction (EF) derived from multiple subjects, which is useful for initial screening of the individual. FIG. 2 is useful in understanding the relationship between the MDST(−C) measured at time point D and ejection fraction, which is a known indicator of the presence or absence of LVD.

It is seen from a consideration of FIGS. 1 and 2 that the MDST(D−C) for the non-LVD individual at time point D, here designated as NLVD-D, is typically 0.16, which is well within the known range of non-LVD patients, while the MDST(D−C) for the LVD individual at time point D, here designated as LVD-D, is typically −0.075, well within the known range of LVD patients.

It is appreciated that by employing the system of FIG. 1 and reaching a conclusion which is diagrammed in FIG. 2, preliminary screening and diagnosis of whether a person suffers from LVD is generally complete.

A preferred next step is to ascertain an ejection fraction (EF) for a person who has been found to suffer from LVD. The ejection fraction is important for immediate and longer term treatment and for monitoring.

In accordance with a preferred embodiment of the present invention, the ejection fraction is determined by employing an algorithm of which the following equation is a current preferred example:

Ejection Fraction(EF)(%)=K₁+K₂×MDST(D−C)+K₃×A+K₄×MF+K₅×W+K₆×HT+K₇×DTDE+K₈×DPEM+K₉×LVD I.

Where:

K₁-K₉ are constants;

MDST(D−C) is the Measured Differential Skin Temperature at point D;

A is Age in Years;

MF is 0 for males and 1 for females;

W is Weight in Kilograms;

HT is Height in Centimeters;

DTDE is Distance in meters Traveled during Physical Exertion;

DPEM is Duration of Physical Exertion in Minutes; and

LVD is 0 for non-LVD and 1 for LVD.

Preferably, K₁ is approximately 26, K₂ is approximately −1.5, K₃ is approximately −0.1, K₄ is approximately 1.93, K₅ is approximately −0.3, K₆ is approximately 0.3, K₇ is approximately −0.03, K₈ is approximately 2.6, K₉ is approximately −30.

Thus, for an LVD positive patient having the following test parameters, the Ejection Fraction (EF) calculated in accordance with a preferred embodiment of the present invention is 34.51%. The EF which was measured by a conventional echocardiogram was 35%.

MDST(D−C)=−0.0625;

Age=55;

Sex=1;

Weight=55;

Height=157;

DTDE=419.77;

DPEM=9.22; and

LVD=1.

Further in accordance with a preferred embodiment of the present invention, the ejection fraction is determined by employing an algorithm of which the following equation is a current preferred example:

Ejection Fraction(EF)(%)=K₁+K₂×MDST(D−C)+K₃×A+K₄×MF+K₅×W+K₆×HT+K₇×DTDE+K₈×DPEM+K₉×LVD+K₁₀×SBP+K₁₁×DBP+K₁₂×TEMP II.

Where:

K₁-K₁₂ are constants;

MDST(D−C) is the Measured differential skin temperature at point D;

A is Age in Years;

MF is 0 for males and 1 for females;

W is Weight in Kilograms;

HT is Height in Centimeters;

DTDE is Distance in meters traveled during Physical Exertion;

DPEM is Duration of Physical Exertion in Minutes;

LVD is 0 for non-LVD and 1 for LVD;

SBP is Systolic Blood Pressure in mm HG;

DBP is Diastolic Blood Pressure in mm HG; and

TEMP is Oral Temperature in ° C.

Preferably K₁ is approximately −26, K₂ is approximately −7, K₃ is approximately −0.05, K₄ is approximately 1.3, K₅ is approximately −0.2, K₆ is approximately 0.2, K₇ is approximately −0.05, K₈ is approximately 3.6, K₉ is approximately −32, K₁₀ is approximately 0.05, K₁₁ is approximately 0.1, K₁₂ is approximately 1.3.

Thus, in this case for the same LVD positive patient having the following test parameters, the Ejection Fraction (EF) calculated in accordance with a preferred embodiment of the present invention is 34.63%. The EF which was measured by a conventional echocardiogram was 35%.

MDST(D−C)=−0.0625;

Age=55;

Sex=1;

Weight=55;

Height=157;

DTDE=419.77;

DPEM=9.22;

LVD=1;

SBP=145;

DBP=90; and

Oral Temperature=37.

Additionally, in accordance with a preferred embodiment of the present invention, the ejection fraction is determined by employing an algorithm of which the following equation is a current preferred example:

Ejection Fraction(EF)(%)=K₁+K₂×MDST(D−C)+K₃×A+K₄×MF+K₅×W+K₆×HT+K₇×DTDE+K₈×DPEM+K₉×LVD+K₁₀×SBP+K₁₁×DBP+K₁₂×TEMP+K₁₃×HRC/HRD III.

Where:

K₁-K₁₃ are constants;

MDST(D−C) is the Measured Differential Skin Temperature at point D;

A is Age in Years;

MF is 0 for males and 1 for females;

W is Weight in Kilograms;

HT is Height in Centimeters;

DTDE is Distance in meters Traveled during physical Exertion;

DPEM is Duration of Physical Exertion in Minutes;

LVD is 0 for non-LVD and 1 for LVD;

SBP is Systolic Blood Pressure in mm HG;

DBP is Diastolic Blood Pressure in mm HG;

TEMP is Oral Temperature in ° C.;

HRC is Heart Rate at time point C in Beats Per Minute (BPM); and

HRD is Heart Rate at time point D in BPM.

Preferably, K₁ is approximately 10, K₂ is approximately −3, K₃ is approximately −0.1, K₄ is approximately −0.2, K₅ is approximately −0.2, K₆ is approximately 0.2, K₇ is approximately −0.05, K₈ is approximately 3.3, K₉ is approximately −31, K₁₀ is approximately 0.1, K₁₁ is approximately 0.01, K₁₂ is approximately 0.4, K₁₃ is approximately −1.

Thus, in this case for the same LVD positive patient having the following test parameters, the Ejection Fraction (EF) calculated in accordance with a preferred embodiment of the present invention is 34.72%. The EF which was measured by a conventional echocardiogram was 35%.

MDST(D−C)=−0.0625;

Age=55;

Sex=1;

Weight=55;

Height=157;

DTDE=419.77;

DPEM=9.22;

LVD=1;

SBP=145;

DBP=90;

Oral Temperature=37; and

HRC/HRD=1.35.

It is appreciated that algorithm I is the most general of the three examples presented above and algorithm II adds parameters to algorithm I and thus presumably provides a more accurate calculation of EF than algorithm I.

Similarly, algorithm III adds parameters to algorithm II and thus presumably provides a more accurate calculation of EF than either of algorithms I or II.

It is further appreciated that the constants which appear in the examples above are based on a limited sample of test subjects and may change or have greater resolution as more subjects are tested.

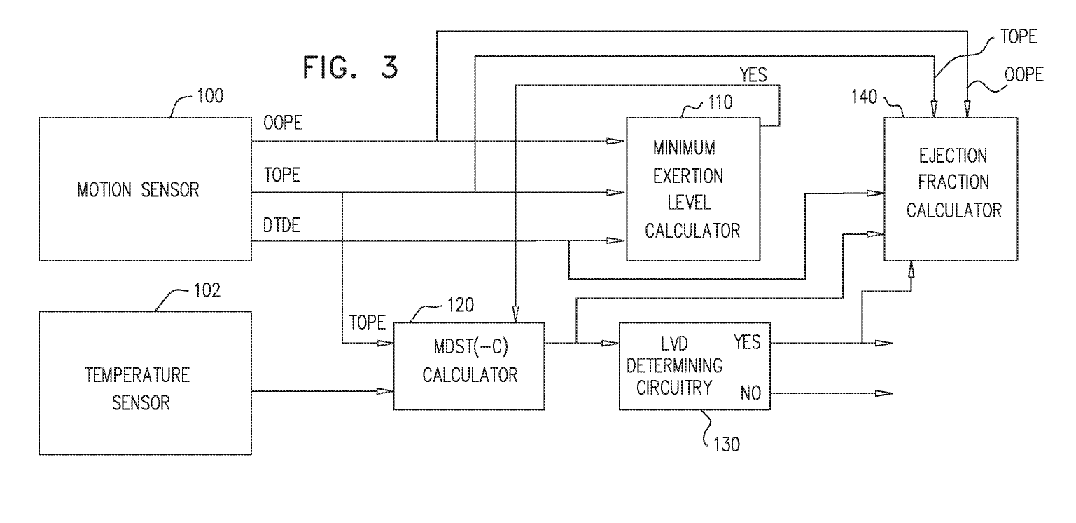

Reference is now made to FIG. 3, which is a simplified functional block diagram of the system of FIG. 1 having the EF calculation functionality described above.

Preferably, motion sensor 100 provides outputs indicating ONSET OF PHYSICAL EXERTION (DOPE) (Time Point B), TERMINATION OF PHYSICAL EXERTION (TOPE) (Time Point C) and DISTANCE TRAVELED DURING PHYSICAL EXERTION (DTDE).

A Minimum Exertion Level Calculator 110 preferably receives all of the outputs of motion sensor 100 and provides a binary output to an MDST(−C) Calculator 120, indicating whether a minimum threshold for physical exertion has been exceeded between the OOPE and the TOPE.

Preferably, temperature sensor 102 operates continuously and provides a SKIN TEMPERATURE OUTPUT (STO) to MDST(−C) Calculator 120, which receives the TOPE output from motion sensor 100 as well an output from Minimum Exertion Level Calculator 110 indicating that at least an acceptable minimum level of Physical Exertion took place between time points B and C and calculates the difference in skin temperature between the time point C indicated by the TOPE output, corresponding to termination of physical exertion, and time point D a predetermined time thereafter, typically 140 seconds. It is appreciated that the time duration separating time points D and C is based on a limited sample of test subjects and may change or have greater resolution as more subjects are tested. The MDST(−C) Calculator 120 provides an MDST(D−C) output to LVD Determining Circuitry 130, which preferably provides a binary output indicating whether there appears to be an LVD condition or not. Additionally or alternatively, the LVD Determining Circuitry 130 may provide an analog output indicating a degree of certainty and/or degree of severity of an LVD condition.

An Ejection Fraction Calculator 140 receives the MDST(D−C) output from MDST(−C) calculator 120, the output of the LVD determining circuitry 130 as well as the OOPE, TOPE and DTDE outputs of motion sensor 100. The OOPE, TOPE and DTDE outputs of motion sensor 100 are provided to the Ejection Fraction Calculator 140 and enable the Ejection Fraction Calculator 140 to calculate the DPEM parameter appearing in algorithm examples I, II and III. The Ejection Fraction Calculator 140 also preferably receives data regarding the person undergoing testing including the following parameters, which appear in algorithm examples I, II and III: Age in Years; Sex, Weight in Kilograms & Height in Centimeters.

Further in accordance with a preferred embodiment of the present invention, the Ejection Fraction Calculator 140 also receives data regarding the person undergoing testing including the following parameters, which appear in algorithm examples II and III: Systolic and Diastolic Blood Pressure & oral temperature.

Additionally, in accordance with a preferred embodiment of the present invention, the Ejection Fraction Calculator 140 also receives data regarding the person undergoing testing including the following parameters, which appear in algorithm example III: Heart Rate. Heart rate data may be provided by any suitable heart rate sensing device.

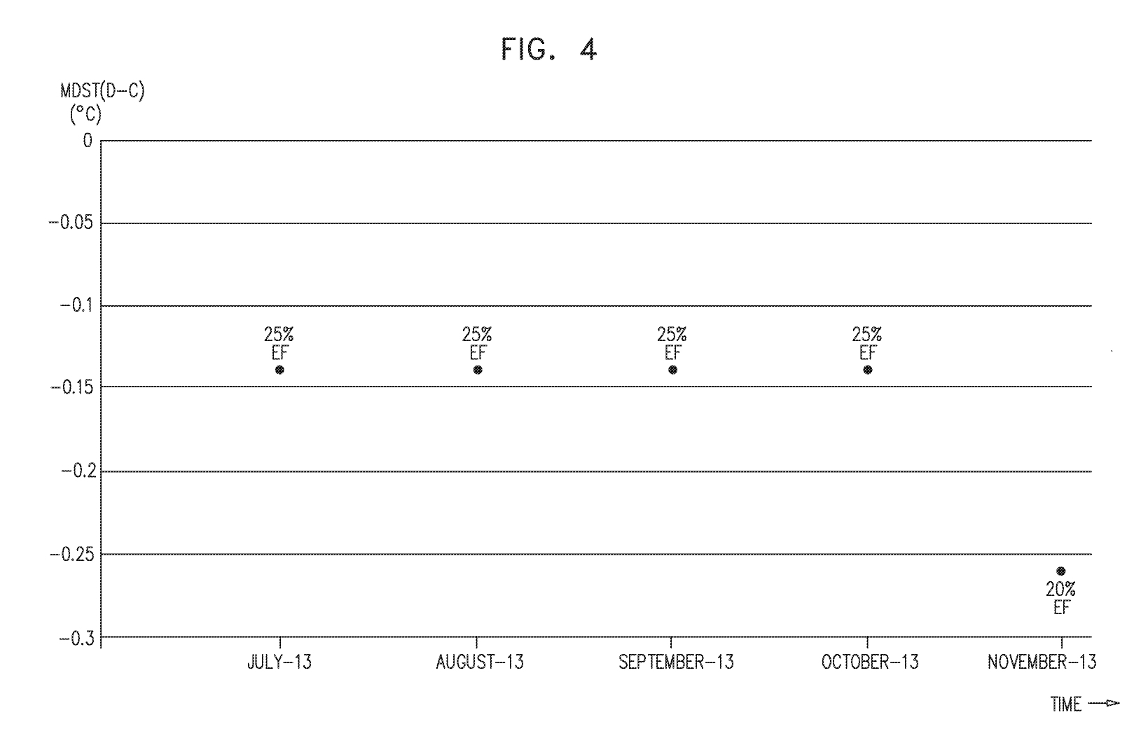

Reference is now made to FIG. 4, which is a simplified illustration of the values of MDST(D−C) for a given individual monitored on multiple occasions, which is useful for monitoring of the individual. In the example shown in FIG. 4, it is seen that although the MDST(D−C) for the individual remains stable and constant at measuring points in July, August, September and October, 2013, it falls precipitously in November, 2013, indicating the probability of a condition which requires clinical intervention.

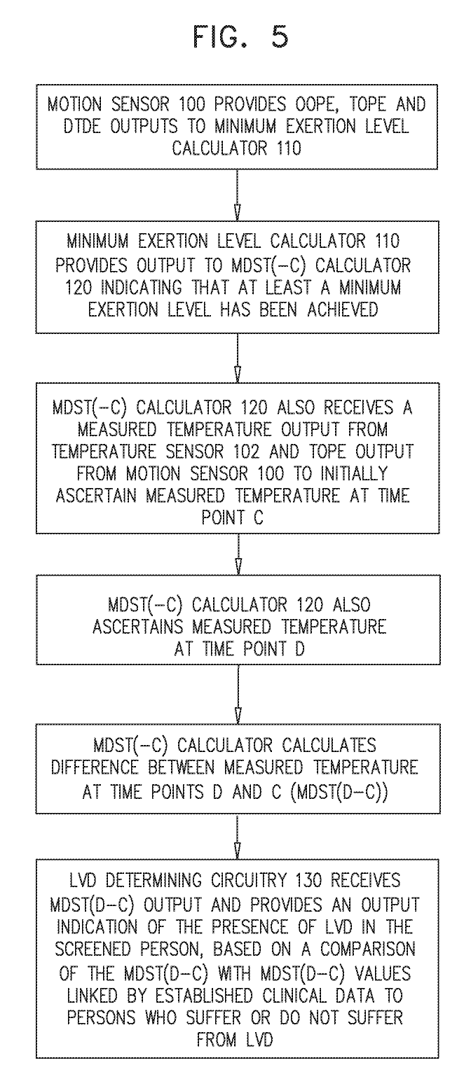

Reference is now made to FIG. 5, which is a simplified flowchart illustrating operation of the system of FIGS. 1-3 for screening. As seen in FIG. 5, the motion sensor 100 provides the OOPE, TOPE and DTDE outputs to Minimum Exertion Level Calculator 110, which provides an output to MDST(−C) Calculator 120 indicating that at least a minimum exertion level has been achieved. It is appreciated that DTDE is a cumulative metric which increases over the time duration of physical exertion. It is further appreciated that alternatively physical exertion may not consist of walking or running, wherein a cumulative distance metric is appropriate, and may instead consist of a different type of physical exertion, having a different cumulative metric, which may be used instead of DTDE.

This output is used by the MDST(−C) Calculator 120, which receives a measured temperature output from the temperature sensor 102 and the TOPE output from motion sensor 100 to initially ascertain the measured temperature at time point C and the measured temperature at time point D thereafter. MDST(−C) calculator 120 calculates the difference between the measured temperature at time points D and C, also referred to as MDST(D−C).

The MDST(D−C) output is received by the LVD Determining Circuitry 130, which provides an output indication of the presence of LVD in the screened person, based on a comparison of the MDST(D−C) with MDST(D−C) values linked by established clinical data to persons who suffer or do not suffer from LVD.

The established clinical data used in the LVD Determining Circuitry 130 may represent an undifferentiated sample population or may be grouped specifically by parameters such as age, sex and weight and matched to screened persons having similar parameters.

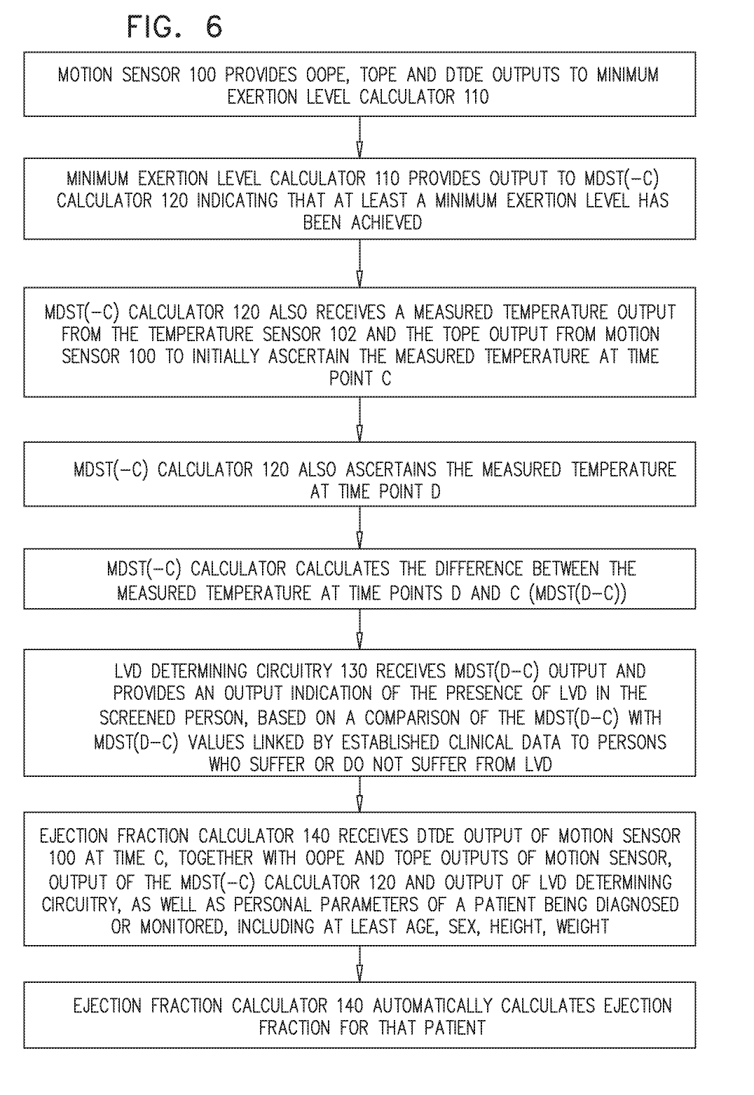

Reference is now made to FIG. 6, which is a simplified flowchart illustrating operation of the system of FIGS. 1 & 4 for EF calculation useful in diagnosis and monitoring. As seen in FIG. 6, the motion sensor 100 provides the OOPE, TOPE and DTDE outputs to Minimum Exertion Level Calculator 110, which provides an output to MDST(−C) Calculator 120 indicating that at least a minimum exertion level has been achieved.

This output is used by the MDST(−C) Calculator 120, which receives a measured temperature output from the temperature sensor 102 and the TOPE output from motion sensor 100 to initially ascertain the measured temperature at time point C and the measured temperature at time point D thereafter. MDST(−C) calculator 120 calculates the difference between the measured temperature at time points D and C, also referred to as MDST(D−C).

The MDST(D−C) output is received by the LVD Determining Circuitry 130, which provides an output indication of the presence of LVD in the screened person, based on a comparison of the MDST(D−C) with MDST(D−C) values linked by established clinical data to persons who suffer or do not suffer from LVD. The established clinical data used in the LVD Determining Circuitry 130 may represent an undifferentiated sample population or may be grouped specifically by parameters such as age, sex and weight and matched to screened persons having similar parameters.

In accordance with a preferred embodiment of the present invention, Ejection Fraction Calculator 140 receives the DTDE output of the motion sensor 100 at time C, together with the OOPE and TOPE outputs of the motion sensor, the output of the MDST(−C) calculator 120 and the output of the LVD Determining Circuitry, as well as personal parameters of a patient being diagnosed or monitored, including at least age, sex, height and weight, and automatically calculates the Ejection Fraction for that patient based on Algorithm Example I hereinabove, wherein the OOPE and TOPE outputs are used by the Ejection Fraction Calculator 140 to calculate DPEM.

Further in accordance with a preferred embodiment of the present invention, Ejection Fraction Calculator 140 additionally receives additional personal parameters including systolic blood pressure, diastolic blood pressure and oral temperature and automatically calculates the Ejection Fraction for that patient based on Algorithm Example II hereinabove.

Still further in accordance with a preferred embodiment of the present invention, Ejection Fraction Calculator 140 additionally receives additional personal parameters including heart rate at time points C and D, systolic blood pressure, diastolic blood pressure and oral temperature and automatically calculates the Ejection Fraction for that patient based on Algorithm Example III hereinabove.

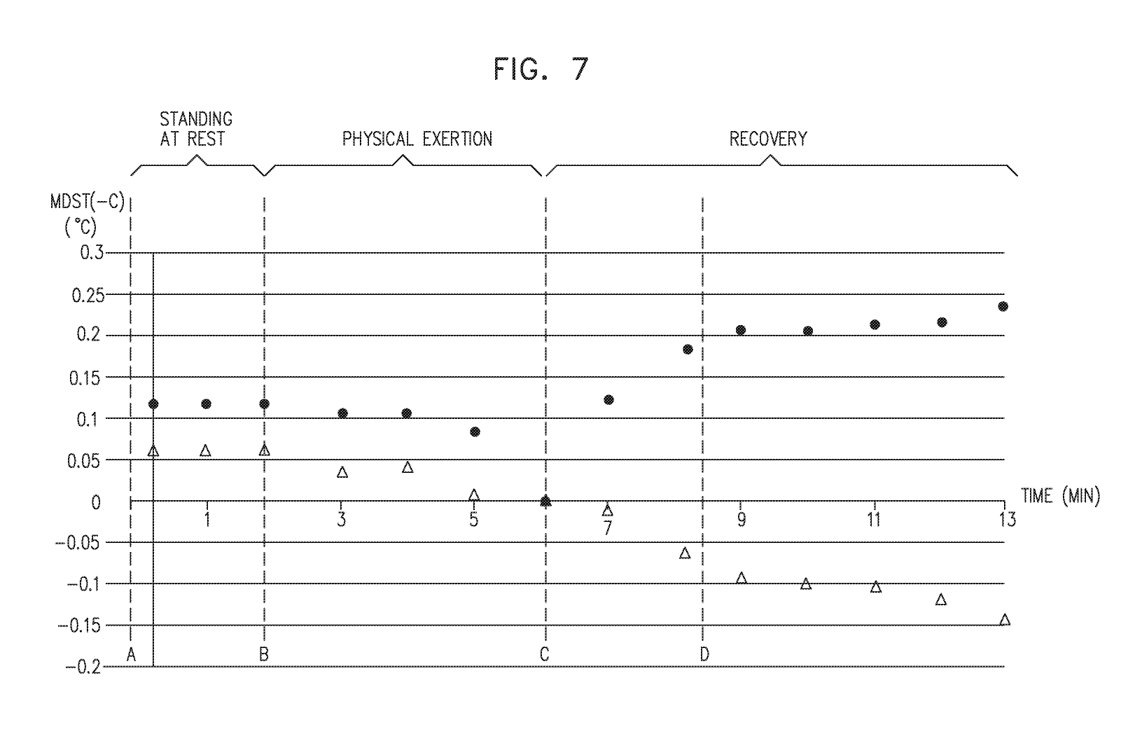

Reference is now made to FIG. 7, which is a simplified diagram showing average experimental MDST(−C) data for non-LVD subjects, indicated by solid dots, and LVD subjects, indicated by triangles. It is seen that in accordance with a preferred embodiment of the present invention, LVD and non-LVD subjects may be readily and automatically distinguished by the increase or decrease in MDST values following time point C.

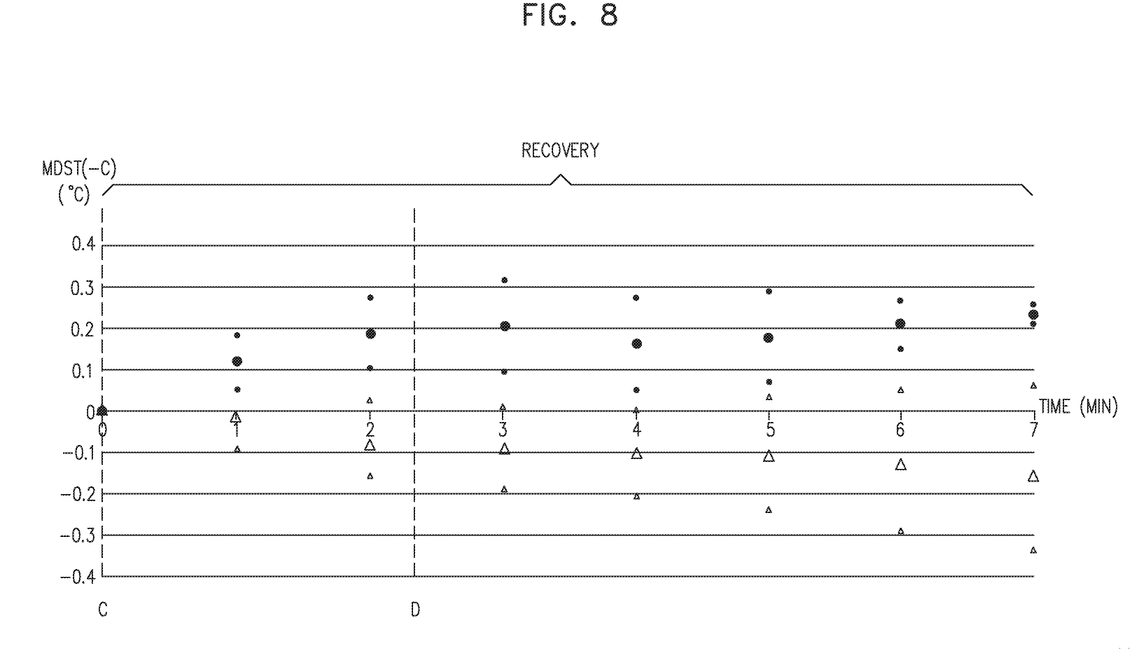

Reference is now made to FIG. 8, which is a simplified diagram showing experimental MDST(−C) data for non-LVD subjects, indicated by solid dots, and LVD subjects, indicated by triangles, from time point C through time point D and therebeyond indicating standard deviations, which are indicated respectively by small solid dots and small triangles.

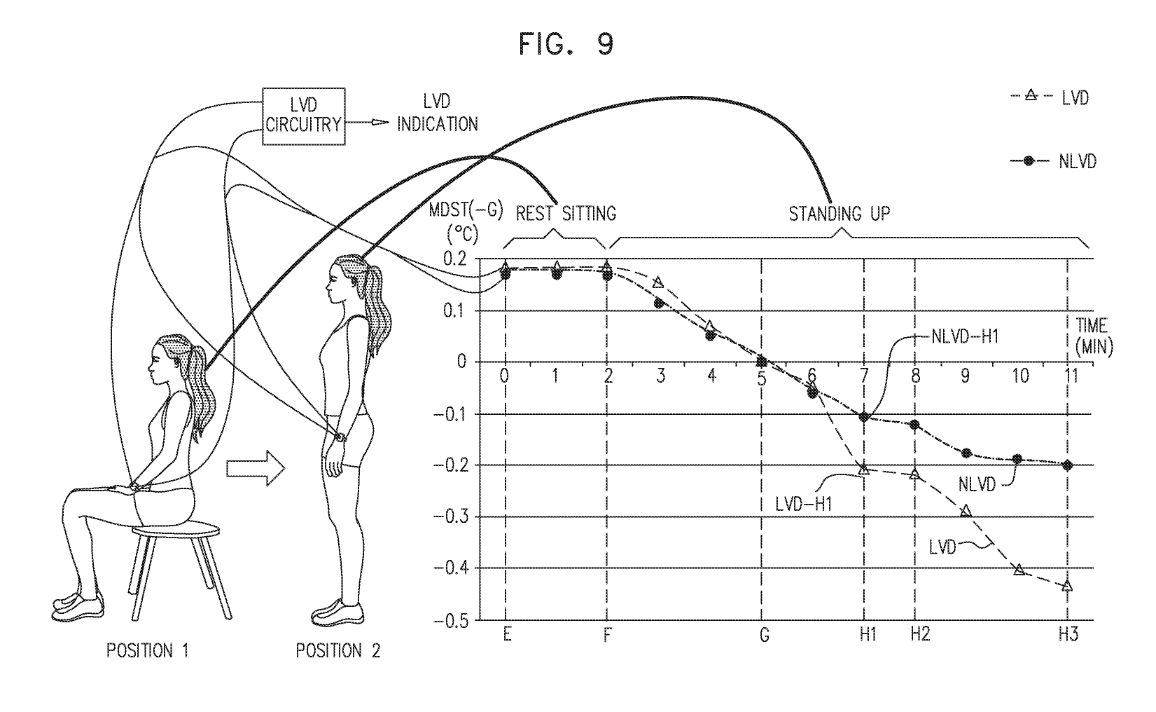

Reference is now made to FIG. 9, which is a simplified illustration of a system which produces an output indication of measured difference in skin temperature (MDST) as a time function of position change for a typical person and provides an indication of at least LVD (Left Ventricular Dysfunction) in accordance with a preferred embodiment of the present invention.

As seen in FIG. 9, a person, herein sometimes referred to as an individual, is shown undergoing a position change, here, for example, standing up after sitting on a chair. The position change of the person is measured by any suitable motion sensor 200, such as a DRM-4000 motion sensor commercially available from Honeywell. The skin temperature of the person is simultaneously measured by a temperature sensor 202, such as an ADT 7420 temperature sensor, commercially available from Analog Devices. The motion sensor 200 may be mounted on a portion of the person's body which is undergoing position change, such as the torso of the person, while the temperature sensor 202 may be mounted on another portion of the person's body, preferably the left wrist of the person. Preferably, both the motion sensor 200 and the temperature sensor 202 are incorporated in a wrist-mounted device, as shown.

Considering now the output of the motion sensor 200, it is seen that the position change of the person is measured from a starting point in time, time 0, designated E, at which the person is sitting down (hereinafter referred to as Position I) and at rest and the onset of position change begins at a point of time designated F when the person stands up (hereinafter referred to as Position II).

The time separation between time points E and F is typically and preferably 2 minutes. A further measuring point in time, typically 3 minutes following time point F, is designated as time point G. At least one of three alternative further measuring points in time, designated as time points H1, H2 and H3, respectively, are established typically at 2 minutes, 3 minutes and 6 minutes following time point G.

Considering now the output of the temperature sensor 202, it is noted that the graph indicates the difference calculated by subtracting the skin temperature at time point G from the sensed skin temperature at a given time on the graph. The graph of the output of temperature sensor 202 is thus appreciated to be a computed graph which is only provided following time point G.

It is seen that for a non-LVD individual, the measured skin temperature minus the measured skin temperature at time point G, herein designated by reference MDST(−G) (Measured differential skin temperature relative to point G) is typically approximately 0.17° C. between time points E and F and then falls, approximately three minutes after time point F generally linearly to zero at time point G. For a typical non-LVD individual, immediately following position change at time point F, the MDST(−G) continues to decrease as shown to time point H2 and typically the decrease becomes less steep therebeyond. The MDST(−G) for a non-LVD individual is designated in FIG. 9 by NLVD.

It is seen that for an individual suffering from LVD, the measured skin temperature minus the measured skin temperature at time point G, herein designated by reference MDST(−G) (Measured differential skin temperature relative to point G) is typically approximately 0.18° C. between time points E and F and then falls after time point F to zero at time point G. For a typical individual suffering from LVD, following position change at time point F, the MDST(−G) continues to decrease as shown for about one minute following time point G. Immediately thereafter the MDST(−G) decreases at an increased rate. The MDST(−G) for an LVD individual is designated in FIG. 9 by LVD.

Appreciation of utilization of the foregoing distinction between MDST(−G) for non-LVD individuals and for LVD individuals are particular features of the present invention.

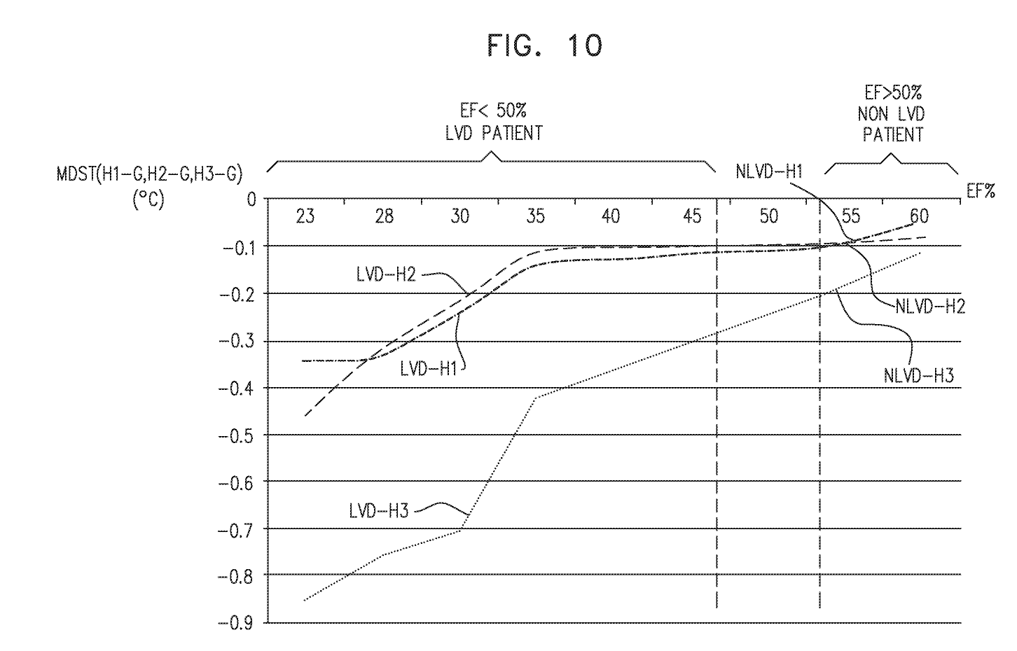

Reference is now made to FIG. 10, which is a simplified illustration of the values of MDST(−G) measured at various time points designated by H1, H2 & H3 vs. ejection fraction (EF) derived from multiple subjects, which is useful for initial screening of individuals. The measured MDST(−G) values for two given individuals, one of whom is an NLVD individual and one of whom is an LVD individual, are marked by NLVD-H1, NLVD-H2 and NLVD-H3 for the non-LVD individual and LVD-H1, LVD-H2 and LVD-H3 for the LVD individual shown in FIG. 10 provide an example useful in understanding the relationship between the MDST(−G) measured at time points H1, H2 & H3 and the ejection fraction (EF), which is a known indicator of the presence or absence of LVD.

It is seen from a consideration of FIGS. 9 and 10 that the MDST(−G) for the non-LVD individual at time point H1, here designated as NLVD-H1, is typically −0.1, which is well within the known range for non-LVD patients, while the MDST(−G) for the LVD individual at time point H1, here designated as LVD-H1 is typically −0.22, well within the known range for LVD patients.

It is appreciated that by employing the system of FIG. 9 and reaching a conclusion which is diagrammed in FIG. 10, screening and preliminary diagnosis of whether a person suffers from LVD is provided.

A preferred next step is to ascertain the ejection fraction (EF) for a person who has been found to suffer from LVD. The ejection fraction is important for immediate and longer term treatment and for monitoring.

In accordance with a preferred embodiment of the present invention, the ejection fraction is determined by employing an algorithm of which the following equation is a current preferred example:

Ejection Fraction(EF)(%)=K₁+K₂×MDST(H2−G)+K₃×A+K₄×MF+K₅×W+K₆×HT+K₇×SBP+K₈×DBP+K₉×TEMP IV.

Where:

K₁-K₉ are constants;

MDST(H2−G) is the Measured Differential Skin Temperature at point H2;

A is Age in Years;

MF is 0 for males and 1 for females;

W is Weight in Kilograms;

HT is Height in Centimeters;

SBP is Systolic Blood Pressure in mm HG;

DBP is Diastolic Blood Pressure in mm HG; and

TEMP is Oral Temperature in ° C.

Preferably, K₁ is approximately −1694, K₂ is approximately 100, K₃ is approximately 0.59, K₄ is approximately 44.2, K₅ is approximately 1.71, K₆ is approximately 2.22, K₇ is approximately −1.41, K₈ is approximately −0.05, K₉ is approximately 44.3.

Thus, for an LVD positive patient having the following test parameters, the Ejection Fraction (EF) calculated in accordance with a preferred embodiment of the present invention is 33.29%. The EF which was measured by a conventional echocardiogram was 35%.

MDST(H2−G)=−0.34;

Age=55;

Sex=1;

Weight=55;

Height=157;

SBP=145;

DBP=90; and

TEMP=37.

Further in accordance with a preferred embodiment of the present invention, the ejection fraction is determined by employing an algorithm of which the following equation is a current preferred example:

Ejection Fraction(EF)(%)=K₁+K₂×MDST(H3−G)+K₃×A+K₄×MF+K₅×W+K₆×HT+K₇×SBP+K₈×DBP+K₉×TEMP V.

Where:

K₁-K₉ are constants;

MDST(H3−G) is the Measured Differential Skin Temperature at point H3;

A is Age in Years;

MF is 0 for males and 1 for females;

W is Weight in Kilograms;

HT is Height in Centimeters;

SBP is Systolic Blood Pressure in mm HG;

DBP is Diastolic Blood Pressure in mm HG; and

TEMP is Oral Temperature in ° C.

Preferably, K₁ is approximately −1065, K₂ is approximately 55.6, K₃ is approximately 0.36, K₄ is approximately 34.1, K₅ is approximately 1.37, K₆ is approximately 1.58, K₇ is approximately −1.10, K₈ is approximately −0.07, K₉ is approximately 29.0.

Thus, in this case for the same LVD positive patient having the following test parameters, the Ejection Fraction (EF) calculated in accordance with a preferred embodiment of the present invention is 36%. The EF which was measured by a conventional echocardiogram was 35%.

MDST(H3−G)=−0.59;

Age=55;

Sex=1;

Weight=55;

Height=157;

SBP=145;

DBP=90; and

TEMP=37.

Additionally, in accordance with a preferred embodiment of the present invention, the ejection fraction is determined by employing an algorithm of which the following equation is a current preferred example:

Ejection Fraction(EF)(%)=K₁+K₂×MDST(H1−G)+K₃×A+K₄×MF+K₅×W+K₆×HT+K₇×SBP+K₈×DBP+K₉×TEMP+K₁₀×LVD VI.

Where:

K₁-K₁₀ are constants;

MDST(H1−G) is the Measured Differential Skin Temperature at point H1;

A is Age in Years;

MF is 0 for males and 1 for females;

W is Weight in Kilograms;

HT is Height in Centimeters;

SBP is Systolic Blood Pressure in mm HG;

DBP is Diastolic Blood Pressure in mm HG;

TEMP is Oral Temperature in ° C.; and

LVD is 0 for non-LVD and 1 for LVD.

Preferably, K₁ is approximately −192, K₂ is approximately 35.5, K₃ is approximately 0.11, K₄ is approximately 4.05, K₅ is approximately 0.33, K₆ is approximately 0.30, K₇ is approximately −0.11, K₈ is approximately 0.03, K₉ is approximately 6.32, K₁₀ is approximately −26.0.

Thus, in this case for the same LVD positive patient having the following test parameters, the Ejection Fraction (EF) calculated in accordance with a preferred embodiment of the present invention is 34.185%. The EF which was measured by a conventional echocardiogram was 35%.

MDST(H1−G)=−0.21;

Age=55;

Sex=1;

Weight=55;

Height=157;

SBP=145;

DBP=90;

TEMP=37; and

LVD=1.

Even further in accordance with a preferred embodiment of the present invention, the ejection fraction is determined by employing an algorithm of which the following equation is a current preferred example:

Ejection Fraction(EF)(%)=K₁+K₂×MSDT-H3+K₃×A+K₄×MF+K₅×W+K₆×HT+K₇×SBP+K₈×DBP+K₉×TEMP+K₁₀×LVD VII.

Where:

K₁-K₁₀ are constants;

MSDT-H3 is the Measured Differential Skin Temperature at point H3;

A is Age in Years;

MF is 0 for males and 1 for females;

W is Weight in Kilograms;

HT is Height in Centimeters;

SBP is Systolic Blood Pressure in mm HG;

DBP is Diastolic Blood Pressure in mm HG;

TEMP is Oral Temperature in ° C.; and

LVD is 0 for non-LVD and 1 for LVD.

Preferably, K₁ is approximately −85.3, K₂ is approximately 14.4, K₃ is approximately 0.07, K₄ is approximately 3.04, K₅ is approximately 0.24, K₆ is approximately 0.19, K₇ is approximately −0.10, K₈ is approximately 0.05, K₉ is approximately 3.77, K₁₀ is approximately −24.7.

Thus, in this case for the same LVD positive patient having the following test parameters, the Ejection Fraction (EF) calculated in accordance with a preferred embodiment of the present invention is 34.5%. The EF which was measured by a conventional echocardiogram was 35%.

MDST(H3−G)=−0.59;

Age=55;

Sex=1;

Weight=55;

Height=157;

SBP=145;

DBP=90;

TEMP=37; and

LVD=1.

It is appreciated that algorithms IV & V are the more general of the four examples presented above and algorithms VI & VII add a parameter to algorithms IV & V and thus presumably provide a more accurate calculation of EF.

It is further appreciated that the constants which appear in the examples above are based on a limited sample of test subjects and may change or have greater resolution as more subjects are tested.

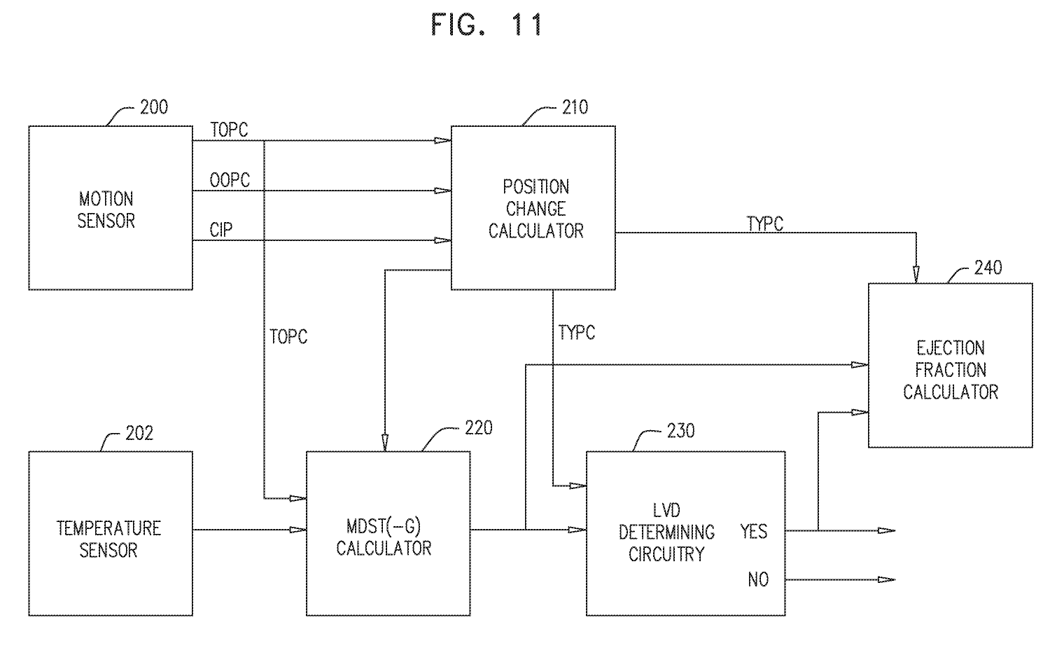

Reference is now made to FIG. 11, which is a simplified functional block diagram of the system of FIG. 9 having the EF calculation functionality described above.

Preferably, motion sensor 200 provides outputs indicating ONSET OF POSITION CHANGE (OOPC), TERMINATION OF POSITION CHANGE (TOPC) (Time Point F) and CHANGE IN POSITION (position 1 to position 2—CIP). The output indicating CIP is typically a signal which represents multidirectional acceleration amplitudes, displacement and angular shifts.

A Position Change Calculator 210 preferably receives all of the outputs of motion sensor 200 and provides a binary output to an MDST(−G) Calculator 220, indicating whether a qualifying position change has been performed by the individual. In addition, the Position Change Calculator 210 provides the type of position change (TYPC) that has been performed by the individual.

Preferably, temperature sensor 202 operates continuously and provides a SKIN TEMPERATURE OUTPUT to MDST(−G) Calculator 220 which calculates the difference in skin temperature between the time point G indicated by the TOPC output, corresponding to position change, and time points H1, H2 & H3 at predetermined times following point G, typically 120, 180, and 360 seconds. It is appreciated that the time duration separating time points H1, H2 & H3 and time point G is based on a limited sample of test subjects and may change or have greater resolution as more subjects are tested. The MDST(−G) Calculator 220 provides an (H1−G), MDST(H2−G) & MDST(H3−G) output to LVD Determining Circuitry 230 and the Position Change Calculator 210 provides a TYPC output to LVD Determining Circuitry 230, which preferably provides a binary output indicating whether there appears to be an LVD condition or not. Additionally or alternatively, the LVD Determining Circuitry 230 may provide an analog output indicating a degree of certainty and/or degree of severity of an LVD condition.

An Ejection Fraction Calculator 240 receives the (H1−G), MDST(H2−G) & MDST(H3−G) outputs from MDST(−G) Calculator 220, the output of the LVD determining circuitry 230 and the TYPC output of the Position Change Calculator 210. The Ejection Fraction Calculator 240 also preferably receives data regarding the person undergoing testing including the following parameters, which appear in algorithm examples IV, V, VI & VII: Age in Years; Sex, Weight in Kilograms, Height in Centimeters, Systolic & Diastolic Blood Pressure in mm Hg, and Oral Temperature in ° C.

Further in accordance with a preferred embodiment of the present invention, the Ejection Fraction Calculator 240 also receives from LVD Determining Circuitry 230 data regarding LVD existence in the person undergoing testing, which appear in algorithm examples VI and VII.

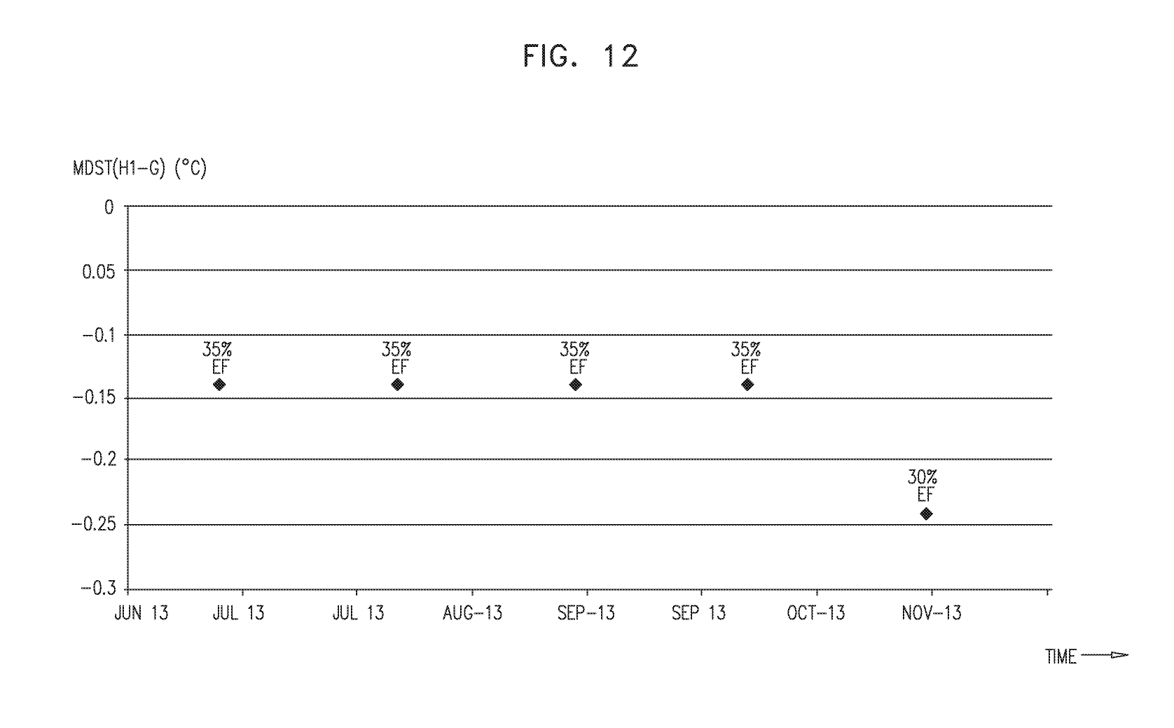

Reference is now made to FIG. 12, which is a simplified illustration of the values of MDST(H1−G) for a given individual monitored on multiple occasions, which is useful for monitoring of the individual. In the example shown in FIG. 12, it is seen that although the MDST(H1−G) values for the individual remain stable and constant at measuring points in July, August, September and October, 2013, it falls precipitously in November, 2013, indicating the probability of a condition which requires clinical intervention.

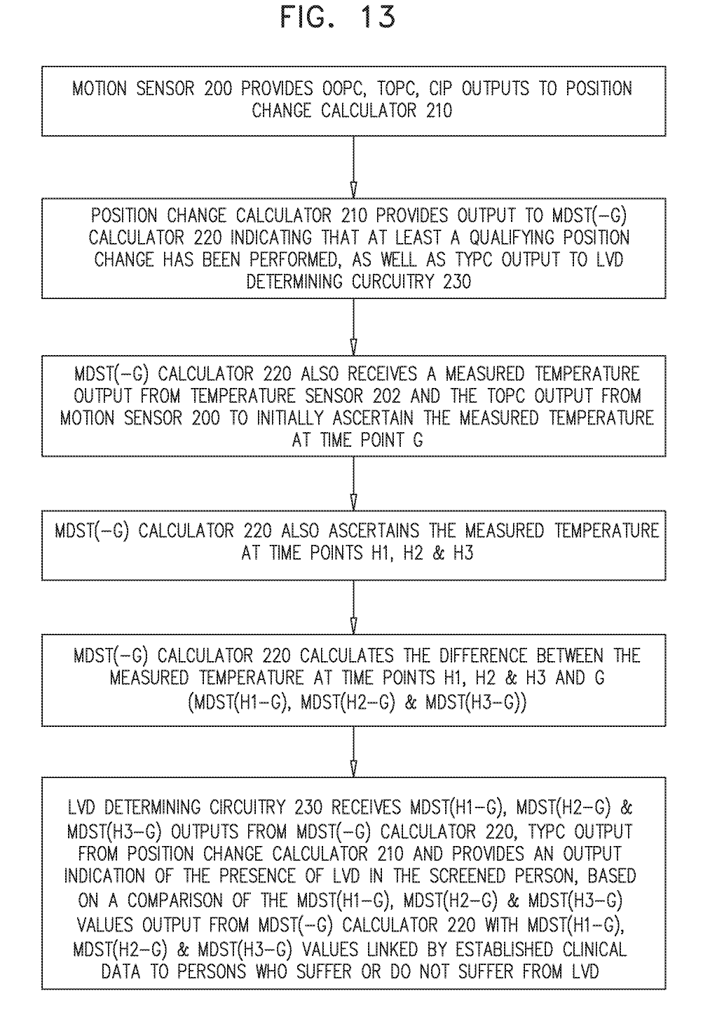

Reference is now made to FIG. 13, which is a simplified flowchart illustrating operation of the system of FIGS. 9 & 10 for screening. As seen in FIG. 13, the motion sensor 200 provides the OOPC, TOPC and CIP outputs to the Position Change Calculator 210, which provides an output to MDST(−G) Calculator 220 indicating that a qualifying position change has been performed by the individual.

This output is used by the MDST(−G) Calculator 220, which receives a measured temperature output from the temperature sensor 202 and the TOPC output from motion sensor 200 to initially ascertain the measured temperature at time point G and the measured temperature at at least one of time points H1, H2 & H3 thereafter. MDST(−G) Calculator 220 calculates the difference between the measured temperature at at least one of time points H1, H2 & H3 and the measured temperature at time point G, also referred to as MDST(H1−G), MDST(H2−G) & MDST(H3−G).

At least one of the MDST(H1−G), MDST(H2−G) & MDST(H3−G) outputs and the TYPC output respectively provided by the MDST(−G) Calculator 220 and the Position Change Calculator 210 are received by the LVD Determining Circuitry 230, which provides an output indication of the presence of LVD in the screened person, based on a comparison of at least one of the MDST(H1−G), MDST(H2−G) & MDST(H3−G) values for the individual with corresponding at least one MDST(H1−G), MDST(H2−G) & MDST(H3−G) values linked by established clinical data to persons who suffer or do not suffer from LVD.

The established clinical data used in the LVD Determining Circuitry 230 may represent an undifferentiated sample population or may be grouped specifically by parameters such as type of position change, age, sex and weight and matched to screened persons having similar parameters.

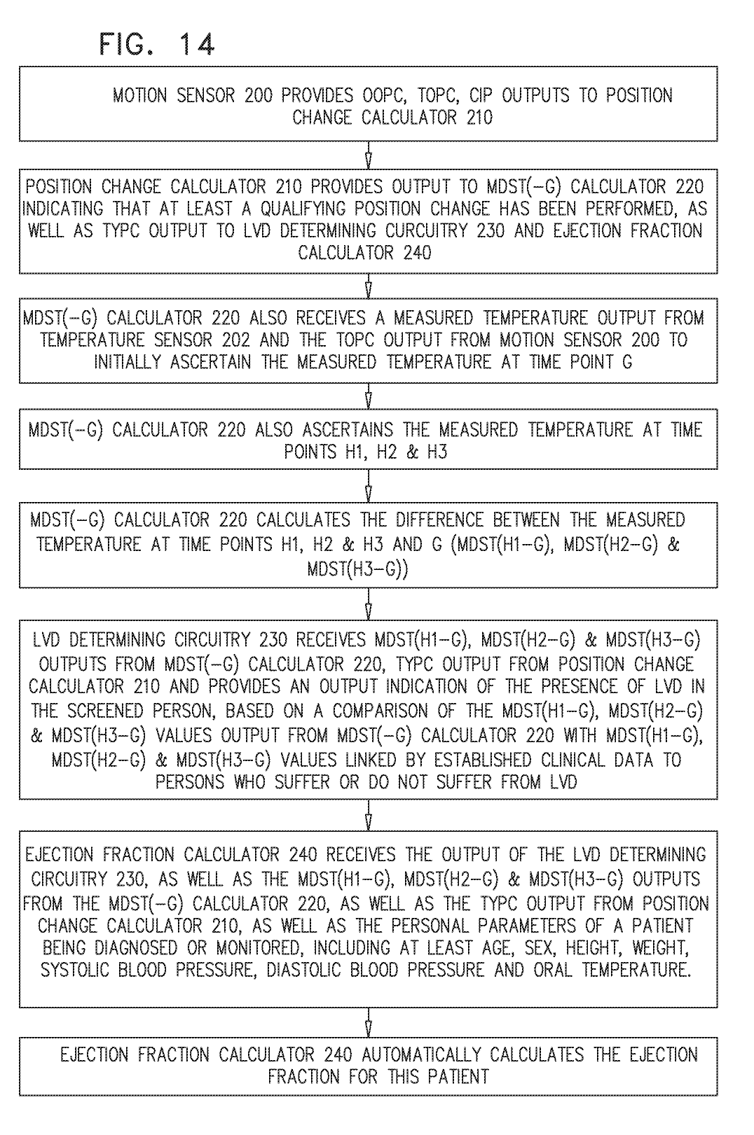

Reference is now made to FIG. 14, which is a simplified flowchart illustrating operation of the system of FIGS. 9, 10 & 12 for EF calculation useful in diagnosis and monitoring. As seen in FIG. 14, the motion sensor 200 provides the OOPC, TOPC and CIP outputs to the Position Change Calculator 210, which provides an output to MDST(−G) Calculator 220 indicating that a qualifying position change has been performed by the individual.

This output is used by the MDST(−G) Calculator 220, which receives a measured temperature output from the temperature sensor 202 and the TOPC output from motion sensor 200 to initially ascertain the measured temperature at time point G and the measured temperature at at least one of time points H1, H2 & H3 thereafter. MDST(−G) Calculator 220 calculates the difference between the measured temperature at at least one of time points H1, H2 & H3 and the measured temperature at time point G, also referred to as MDST(H1−G), MDST(H2−G) & MDST(H3−G).

At least one of the MDST(H1−G), MDST(H2−G) & MDST(H3−G) outputs and the TYPC output respectively provided by the MDST(−G) Calculator 220 and the Position Change Calculator 210 are received by the LVD Determining Circuitry 230, which provides an output indication of the presence of LVD in the screened person, based on a comparison of at least one of the MDST(H1−G), MDST(H2−G) & MDST(H3−G) values of the individual with corresponding at least one of MDST(H1−G), MDST(H2−G) & MDST(H3−G) values linked by established clinical data to persons who suffer or do not suffer from LVD.

The established clinical data used in the LVD Determining Circuitry 230 may represent an undifferentiated sample population or may be grouped specifically by parameters such as type of position change, age, sex and weight and matched to screened persons having similar parameters.

In accordance with a preferred embodiment of the present invention, Ejection Fraction Calculator 240 receives the output of the MDST(−G) Calculator 220 and the output of the LVD Determining Circuitry 230, the TYPC output of Position Change Calculator 210 as well as personal parameters of a patient being diagnosed or monitored, including at least age, sex, height, weight, systolic blood pressure, diastolic blood pressure, oral temperature and automatically calculates the Ejection Fraction for that patient based on Algorithm Examples IV & V hereinabove.

Still further in accordance with a preferred embodiment of the present invention, Ejection Fraction Calculator 240 additionally receives from the LVD Determining Circuitry 230 output indicating the existence of LVD in the patient and automatically calculates the Ejection Fraction for that patient based on Algorithm Examples VI & VII hereinabove.

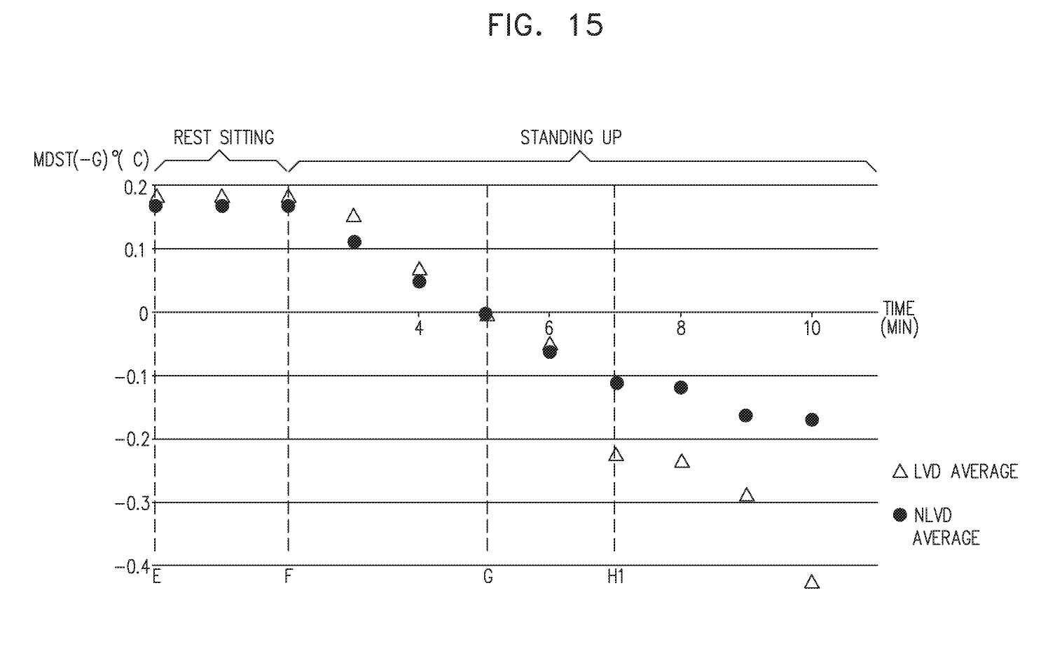

Reference is now made to FIG. 15, which is a simplified diagram showing average experimental MDST(−G) data for non-LVD subjects, indicated by solid dots, and LVD subjects, indicated by triangles. It is seen that in accordance with a preferred embodiment of the present invention, LVD and non-LVD subjects may be readily and automatically distinguished by the magnitude of decrease in MDST(−G) values following time point G.

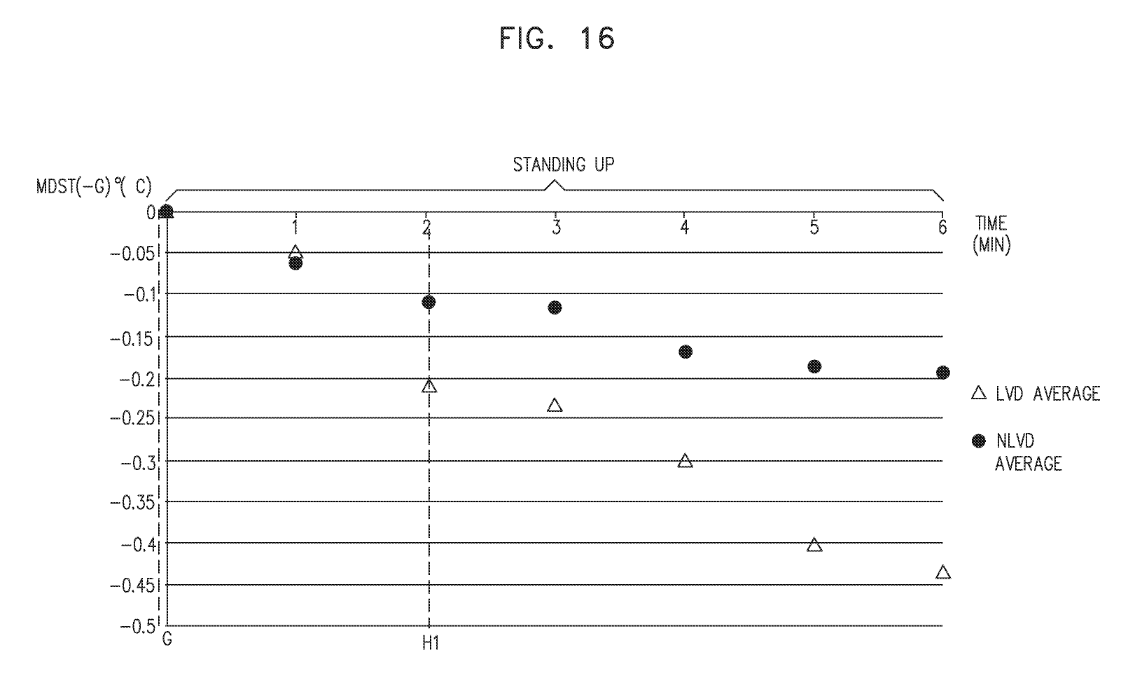

Reference is now made to FIG. 16, which is a simplified diagram showing experimental MDST(−G) data for non-LVD subjects, indicated by solid dots, and LVD subjects, indicated by triangles, from time point G through time points H1, H2 & H3 and therebeyond.

It will be appreciated by persons skilled in the art that the present invention is not limited by what has been particularly shown and described hereinabove but includes generalizations and alternatives thereof which are not shown in the prior art.

1. A system for providing an indication of at least LVD (Left Ventricular Dysfunction), the system comprising:
at least one temperature sensor providing an output indication based on skin temperature at at least one location on a person at a plurality of given times;
at least one body activity sensor providing an output indication of at least termination of body activity;
a time/temperature ascertainer operative to receive inputs from said at least one temperature sensor and from said at least one body activity sensor to provide output indications of said skin temperature at termination of body activity and thereafter; and
a correlator operative to correlate said output indications of said skin temperature at termination of body activity and thereafter with established clinical data relating changes in skin temperature at termination of body activity and thereafter to existence of at least LVD, said correlator providing at least an output indication of at least LVD.

2-3. (canceled)

4. A system for providing an indication of at least LVD according to claim 1 and wherein said at least one temperature sensor and said at least one body activity sensor respectively measure temperature and body activity such that said temperature represents skin temperature at a body region which is less active than a region which is principally undergoing body activity.

5. A system for providing an indication of at least LVD according to claim 1 and wherein said at least one body activity sensor is embodied in a treadmill.

6. A system for providing an indication of at least LVD according to claim 1 and wherein said temperature sensor measures skin temperature on a person's wrist.

7. A system for providing an indication of at least LVD according to claim 1 and wherein said body activity sensor is mounted on a portion of the person's body which is undergoing physical exertion while said temperature sensor is mounted on a portion of the person's body other than that portion undergoing physical exertion.

8-9. (canceled)

10. A system for providing an indication of at least LVD according to claim 1 and wherein:
physical exertion of the person is measured from a starting point in time designated time A at which the person is standing and at rest;
the onset of physical exertion begins at a point in time designated B; and
said physical exertion is terminated at a point in time designated C;
a further measuring point in time, designated time point D, is established following time point C; and
measured differential skin temperature relative to point C (MDST(−C)) increases from time point C to time point D for a non-LVD person.

11. A system for providing an indication of at least LVD according to claim 1 and wherein:
physical exertion of the person is measured from a starting point in time designated time A at which the person is standing and at rest;
the onset of physical exertion begins at a point in time designated B; and
said physical exertion is terminated at a point in time designated C;
a further measuring point in time, designated time point D, is established following time point C; and
measured differential skin temperature relative to point C (MDST(−C)) decreases from time point C to time point D for an LVD person.

12. A system according to claim 1 and also comprising an ejection fraction calculator operative to ascertain the ejection fraction (EF) for said person.

13-18. (canceled)

19. A system according to claim 1 and wherein:
physical exertion of the person is measured from a starting point in time designated time A at which the person is standing and at rest;
the onset of physical exertion begins at a point in time designated B; and
said physical exertion is terminated at a point in time designated C;
a further measuring point in time, designated time point D, is established following time point C; and
said body activity sensor provides outputs indicating ONSET OF PHYSICAL EXERTION (DOPE) (Time Point B), TERMINATION OF PHYSICAL EXERTION (TOPE) (Time Point C) and DISTANCE TRAVELED DURING PHYSICAL EXERTION (DTDE).

20. A system according to claim 19 and also comprising a minimum exertion level calculator receiving said outputs of said body activity sensor and providing an output indicating whether a minimum threshold for physical exertion has been exceeded between the DOPE and the TOPE.

21. A system for providing an indication of at least LVD according to claim 1 wherein:
body activity of the person is measured from a starting point in time designated time E at which the person is sitting and at rest; and
said body activity terminates at a point in time designated time F.

22-24. (canceled)

25. A system for providing an indication of at least LVD according to claim 21 and wherein:
a further measuring point in time, designated reference time point G, is established following time point F; and
a measured differential skin temperature (MDST(−G)) decreases more significantly following time point G for an LVD person than for a non-LVD person.

26-34. (canceled)

35. A system according to claim 25 and wherein said body activity sensor provides outputs indicating ONSET OF POSITION CHANGE (OOPC), TERMINATION OF POSITION CHANGE (TOPC) (Time Point F) and CHANGE IN POSITION (CIP).

36. A system according to claim 35 and also comprising a body position change calculator receiving said outputs of said body activity sensor and providing an output indicating whether a qualifying position change has been performed between the OOPC and the TOPC as well as the TYPE OF POSITION CHANGE (TYPC).

37. A method for providing an indication of at least LVD (Left Ventricular Dysfunction), the method comprising:
sensing a skin temperature of a subject at at least one location on a person at a plurality of given times;
providing a plurality of skin temperature output indications based on said sensing;
sensing body activity of said subject and providing an output indication of at least termination of said body activity;
ascertaining skin temperature of said subject at said termination of body activity and thereafter based on said plurality of skin temperature output indications and said output indication of at least termination of said body activity;
correlating said skin temperature of said subject at said termination of body activity and thereafter with established clinical data relating changes in skin temperature at said termination of body activity and thereafter to existence of at least LVD; and
providing at least an output indication of at least LVD.

38-40. (canceled)

41. A method for providing an indication of at least LVD according to claim 37 wherein:
physical exertion of the person is measured from a starting point in time designated time A at which the person is standing and at rest;
the onset of physical exertion begins at a point in time designated B;
said physical exertion is terminated at a point in time designated C;
and
a further measuring point in time, designated time point D, is established following time point C.

42. A method for providing an indication of at least LVD according to claim 41 wherein said output indication of at least LVD indicates the absence of LVD when measured differential skin temperature relative to point C (MDST(−C)) increases from time point C to time point D.

43. A method for providing an indication of at least LVD according to claim 41 wherein said output indication of at least LVD indicates the presence of LVD when measured differential skin temperature relative to point C (MDST(−C)) decreases from time point C to time point D.

44-50. (canceled)

51. A method for providing an indication of at least LVD according to claim 37 and wherein:
said body activity of the person is measured from a starting point in time designated time E at which the person is sitting and at rest;
said body activity terminates at a point in time designated time F;
a reference time point G is established following time F; and
at least one of three further measuring points in time, designated time points H1, H2 & H3, is established following time point G.

52-53. (canceled)

54. A method for providing an indication of at least LVD according to claim 51 wherein said output indication of at least LVD indicates absence of LVD when measured differential skin temperature relative to point G (MDST(−G)) decreases at a lower rate than a respective predetermined threshold from time point G to at least one of time points H1, H2 and H3.

55. A method for providing an indication of at least LVD according to claim 51 wherein said output indication of at least LVD indicates presence of LVD when measured differential skin temperature relative to point G (MDST(−G)) decreases at a higher rate than a respective predetermined threshold from time point G to at least one of time points H1, H2 and H3.

56-65. (canceled)
